SEQUENCE LISTING (10 sequences)

SEQ ID NO: 1 - protein, 312 aa, Homo sapien [sequence omitted]

SEQ ID NO: 2 - DNA, 939 bp, Homo sapien
atggatccaaacactgtgtcaagctttcaggtagattgctttctttggcatgtccgcaaacgagttgcagaccaagaactaggtgatgccccattccttgatcggcttcgccgagatcagaaatccctaagaggaaggggcagcaccctcggtctggacatcgagacagccacacgtgctggaaagcagatagtggagcggattctgaaagaagaatccgatgaggcacttaaaatgaccatggttggggaggactgcagcccgcactcgcagccctggcaggcggcactggtcatggaaaacgaattgttctgctcgggcgtcctggtgcatccgcagtgggtgctgtcagccgcagcgtgtttccagaactcctacaccatcgggctgggcctgcacagtcttgaggccgaccaagagccagggagccagatggtggaggccagcctctccgtacggcacccagagtacaacagacccttgctcgctaacgacctcatgctcatcaagttggacgaatccgtgtccgagtctgacaccatccggagcatcagcattgcttcgcagtgccctaccgcggggaactcttgcctcgtttctggctggggtctgctggcgaacggcagaatgcctaccgtgctgcagtgcgtgaacgtgtcggtggtgtctgaggaggtctgcagtaagctctatgacccgctgtaccaccccagcatgttctgcgccggcggagggcaagaccagaaggactcctgcaacggtgactctggggggcccctgatctgcaacgggtacttgcagggccttgtgtctttcggaaaagccccgtgtggccaagttggcgtgccaggtgtctacaccaacctctgcaaattcactgagtggatagagaaaaccgtccaggccagtactagtggccaccatcaccatcaccattaa

SEQ ID NO: 3 - protein, 312 aa, Homo sapien
MDPNTVSSFQVDCFLWHVRKRVADQELGDAPFLDRLRRDQKSLRGRGSTLGLDIETATRAGKQIVERILKEESDEALKMTMVGEDCSPHSQPWQAALVMENELFCSGVLVHPQWVLSAAHCFQNSYTIGLGLHSLEADQEPGSQMVEASLSVRHPEYNRPLLANDLMLIKLDESVSESDTIRSISIASQCPTAGNSCLVSGWGLLANGRMPTVLQCVNVSVVSEEVCSKLYDPLYHPSMFCAGGGQDQKDSCNGDSGGPLICNGYLQGLVSFGKAPCGQVGVPGVYTNLCKFTEWIEKTVQASTSGHHHHHH

SEQ ID NO: 4 - DNA, 939 bp, Homo sapien
atggatccaaacactgtgtcaagctttcaggtagattgctttctttggcatgtccgcaaacgagttgcagaccaagaactaggtgatgccccattccttgatcggcttcgccgagatcagaaatccctaagaggaaggggcagcaccctcggtctggacatcgagacagccacacgtgctggaaagcagatagtggagcggattctgaaagaagaatccgatgaggcacttaaaatgaccatggttggggaggactgcagcccgcactcgcagccctggcaggcggcactggtcatggaaaacgaattgttctgctcgggcgtcctggtgcatccgcagtgggtgctgtcagccgcacactgtttccagaactcctacaccatcgggctgggcctgcacagtcttgaggccgaccaagagccagggagccagatggtggaggccagcctctccgtacggcacccagagtacaacagacccttgctcgctaacgacctcatgctcatcaagttggacgaatccgtgtccgagtctgacaccatccggagcatcagcattgcttcgcagtgccctaccgcggggaactcttgcctcgtttctggctggggtctgctggcgaacggcagaatgcctaccgtgctgcagtgcgtgaacgtgtcggtggtgtctgaggaggtctgcagtaagctctatgacccgctgtaccaccccagcatgttctgcgccggcggagggcaagaccagaaggactcctgcaacggtgactctggggggcccctgatctgcaacgggtacttgcagggccttgtgtctttcggaaaagccccgtgtggccaagttggcgtgccaggtgtctacaccaacctctgcaaattcactgagtggatagagaaaaccgtccaggccagtactagtggccaccatcaccatcaccattaa

SEQ ID NO: 5 - protein, 232 aa, Homo sapien
  VARIANT 42: Xaa = Any Amino Acid
NSARAHSQPWQAALVMENELFCSGVLVHPQWVLSAAHCFQKXVQSSYTIGLGLHSLEADQEPGSQMVEASLSVRHPEYNRPLLANDLMLIKLDESVSESDTIRSISIASQCPTAGNSCLVSGWGLLANGRMPTVLQCVNVSVVSEEVCSKLYDPLYHPSMFCAGGGQDQKDSCNGDSGGPLICNGYLQGLVSFGKAPCGQVGVPGVYTNLCKFTEWIEKTVPGQLTLGTGNP

SEQ ID NO: 6 - protein, 248 aa, Homo sapien
  VARIANT 113, 128, 132: Xaa = Any Amino Acid
MWFLVLCLALSLGGTGAAPPIQSRIVGGWECEQHSQPWQAALVMENELFCSGVLVHPQWVLSAAHCFQNSYTIGLGLHSLEADQEPGSQMVEASLSVRHPEYNRPLLANDLMXIKLDESVSESDNIRXISIXSQCPTAGNFCLVSGWGLLANGRMPTVLQCVNVSVVSEEVCSKLYDPLYHPSMFCAGGGQDQKDSCNGDSGGPLICNGYLQGLVSFGKAPCGQVGVPGVYTNLCKFTEWIEKTVQAS

SEQ ID NO: 7 - protein, 254 aa, Homo sapien
MATAGNPWGWFLGYLILGVAGSLVSGSCSQIINGEDCSPHSQPWQAALVMENELFCSGVLVHPQWVLSAAHCFQNSYTIGLGLHSLEADQEPGSQMVEASLSVRHPEYNRPLLANDLMLIKLDESVSESDTIRSISIASQCPTAGNSCLVSGWGLLANGRMPTVLQCVNVSVVSEEVCSKLYDPLYHPSMFCAGGGHDQKDSCNGDSGGPLICNGYLQGLVSFGKAPCGQVGVPGVYTNLCKFTEWIEKTVQAS

SEQ ID NO: 8 - protein, 226 aa, Homo sapien
EFHCVGEDCSPHSQPWQAALVMENELFCSGVLVHPQWVLSAAHCFQNSYTIGLGLHSLEADQEPGSQMVEASLSVRHPEYNRPLLANDLMLIKLDESVSESDTIRSISIASQCPTAGNSCLVSGWGLLANGRMPTVLQCVNVSVVSEEVCSKLYDPLYHPSMFCAGGGQDQKDSCNGDSGGPLICNGYLQGLVSFGKAPCGQVGVPGVYTNLCKFTEWIEKTVQAS

SEQ ID NO: 9 - protein, 231 aa, Homo sapien
MMVGEDCSPHSQPWQAALVMENELFCSGVLVHPQWVLSAAACFQNSYTIGLGLHSLEADQEPGSQMVEASLSVRHPEYNRPLLANDLMLIKLDESVSESDTIRSISIASQCPTAGNSCLVSGWGLLANGRMPTVLQCVNVSVVSEEVCSKLYDPLYHPSMFCAGGGQDQKDSCNGDSGGPLICNGYLQGLVSFGKAPCGQVGVPGVYTNLCKFTEWIEKTVQASGHHHHHH

SEQ ID NO: 10 - DNA, 696 bp, Homo sapien
atgatggttggggaggactgcagcccgcactcgcagccctggcaggcggcactggtcatggaaaacgaattgttctgctcgggcgtcctggtgcatccgcagtgggtgctgtcagccgcagcgtgtttccagaactcctacaccatcgggctgggcctgcacagtcttgaggccgaccaagagccagggagccagatggtggaggccagcctctccgtacggcacccagagtacaacagacccttgctcgctaacgacctcatgctcatcaagttggacgaatccgtgtccgagtctgacaccatccggagcatcagcattgcttcgcagtgccctaccgcggggaactcttgcctcgtttctggctggggtctgctggcgaacggcagaatgcctaccgtgctgcagtgcgtgaacgtgtcggtggtgtctgaggaggtctgcagtaagctctatgacccgctgtaccaccccagcatgttctgcgccggcggagggcaagaccagaaggactcctgcaacggtgactctggggggcccctgatctgcaacgggtacttgcagggccttgtgtctttcggaaaagccccgtgtggccaagttggcgtgccaggtgtctacaccaacctctgcaaattcactgagtggatagagaaaaccgtccaggccagtggccaccatcaccatcaccattaa

(19) United States
(12) Patent Application Publication
(10) Pub. No.: US 2003/0143240 A1
(43) Pub. Date: Jul. 31, 2003

(54) Prostase protein vaccine comprising derivatised thiol residues and methods for producing said antigen

(76) Inventors: Teresa Elisa Virginia Cabezon-Silva, Rixensart (BE); Philippe Jean Gervais Ghislain Permanne, Rixensart (BE)

Correspondence Address: SMITHKLINE BEECHAM CORPORATION, CORPORATE INTELLECTUAL PROPERTY-US, UW2220, P. O. BOX 1539, KING OF PRUSSIA, PA, 19406-0939, US

(21) Appl. No.: 10/312,089

(22) PCT Filed: Jun. 21, 2001
(86) PCT No.: PCT/EP01/07081

(51) Int. Cl. A61K 39/00; C07H 21/04; C12P 21/02; C12N 5/06; C07K 14/47

(52) U.S. Cl. 424/185.1; 435/69.3; 435/320.1; 435/325; 530/350; 536/23.2

(57) ABSTRACT

The present invention relates to chemically modified prostase derivatives, fragments and homologues thereof. Such antigens may be formulated to provide vaccines for the treatment of prostate tumours. Methods for purifying prostase protein and homologues are also provided.

The present invention relates to protein derivatives of a protein known as prostase, a prostate-specific serine protease, to methods for their purification and manufacture, and also to pharmaceutical compositions containing such derivatives, and to their use in medicine. In particular such derivatives find utility in cancer vaccine therapy, particularly prostate cancer vaccine therapy and diagnostic agents for prostate tumours.

In particular the derivatives of the invention include chemically modified prostase protein wherein the antigen's disulphide bridges are reduced and the resulting thiols blocked. Additionally, genetically modified prostase protein and fusion proteins comprising prostase linked to an immunological or an expression enhancer fusion partner containing such blocked thiols are contemplated by the present invention.

The present invention also provides methods for purifying the prostase derivatives and for formulating vaccines for immunotherapeutically treating prostate cancer patients and prostase-expressing tumours other than prostate tumours, prostatic hyperplasia, and prostate intraepithelilial neoplasia (PIN).

Prostate cancer is the most common cancer among males, with an estimated incidence of 30% in men over the age of 50. Overwhelming clinical evidence shows that human prostate cancer has the propency to metastasise to bone, and the disease appears to progress inevitably from androgen dependent to androgen refractory status, leading to increased patient mortality (Abbas F., Scardino P. “The Natural History of Clinical Prostate Carcinoma.” In Cancer (1997); 80:827-833). This prevalent disease is currently the second leading cause of cancer death among men in the US.

Despite considerable research into therapies for the disease, prostate cancer remains difficult to treat. Currently, treatment is based on surgery and/or radiation therapy, but these methods are ineffective in a significant percentage of cases (Frydenberg M., Stricker P., Kaye K. “Prostate Cancer Diagnosis and Management” The Lancet (1997); 349:1681-1687). Several tumour-associated antigens are already known. Many of these antigens may be interesting targets for immunotherapy, but are either not fully tumour-specific or are closely related to normal proteins, and hence bear with them the risk of organ-specific auto-immunity, once targeted by a potent immune response. When an auto-immune response to non-crucial organs can be tolerated, auto-immunity to heart, intestine and other crucial organs could lead to unacceptable safety profiles. Some previously identified prostate specific proteins like prostate specific antigen (PSA) and prostatic acid phosphatase (PAP), prostate-specific membrane antigen (PSMA) and prostate stem cell antigen (PSCA) have limited therapeutic potential and moreover are not always correlated with the presence of prostate cancer or with the level of metastasis (Pound C., Partin A., Eisenberg M. et al. “Natural History of Progression after PSA Elevation following Radical Prostatectomy.” In Jama (1999); 281:1591-1597) (Bostwick D., Pacelli A., Blute M. et al. “Prostate Specific Membrane Antigen Expression in Prostatic Intraepithelial Neoplasia and Adenocarcinoma.” In Cancer (1998); 82:2256-2261).

The existence of tumour rejection mechanisms has been recognised since several decades. Tumour antigens, though encoded by the genome of the organism and thus theoretically not recognized by the immune system through the immune tolerance phenomenon, can occasionally induce immune responses detectable in cancer patients. This is evidenced by antibodies or T cell responses to antigens expressed by the tumour (Xue B H., Zhang Y., Sosman J. et al.

“Induction of Human Cytotoxic T-Lymphocytes Specific for Prostate-Specific Antigen.” In Prostate (1997); 30(2):73-78). When relatively weak anti-tumour effects can be observed through the administration of antibodies recognizing cell surface markers of tumour cells, induction of strong T cell responses to antigens expressed by tumour cells can lead to complete regression of established tumours in animal models (mainly murine).

It is now recognised that the expression of tumour antigens by a cell is not sufficient for induction of an immune response to these antigens. Initiation of a tumour rejection response requires a series of immune amplification phenomena dependent on the intervention of antigen presenting cells, responsible for delivery of a series of activation signals.

Prostase is a prostate-specific serine protease (trypsin-like), 254 amino acid-long, with a conserved serine protease catalytic triad H-D-S and a amino-terminal pre-propeptide sequence, indicating a potential secretory function (P. Nelson, Lu Gan, C.

Ferguson, P. Moss, R. Gelinas, L. Hood & K. Wand, “Molecular cloning and characterisation of prostase, an androgen-regulated serine protease with prostate restricted expression, In Proc. Natl. Acad. Sci. USA (1999) 96, 3114-3119). A putative glycosylation site has been described. The predicted structure is very similar to other known serine proteases, showing that the mature polypeptide folds into a single domain. The mature protein is 224 amino acids-long, with one A2 epitope shown to be naturally processed.

Prostase nucleotide sequence and deduced polypeptide sequence and homologs are disclosed in Ferguson, et al. (Proc. Natl. Acad. Sci. USA 1999, 96, 3114-3119) and in International Patent Applications. No. WO 98/12302 (and also the corresponding granted patent U.S. Pat. No. 5,955,306), WO 98/20117 (and also the corresponding granted patents U.S. Pat. No. 5,840,871 and U.S. Pat. No. 5,786,148) (prostate-specific kallikrein) and WO 00/04149 (P703P).

The present invention provides chemically modified prostase protein derivatives, and fragments and homologues thereof, wherein prostase comprises derivatised thiol residues. Such derivatives are suitable for use in therapeutic vaccine formulations which are suitable for the treatment of a prostate tumours and prostase-expressing tumours.

Prostase fragments of the invention will be of at least about 10 consecutive amino acids, preferably about 20, more preferably about 50, more preferably about 100, more preferably about 150 contiguous amino acids selected from the amino acid sequences as shown in SEQ ID N°5 or SEQ ID N°6 or SEQ ID N°7 or SEQ ID N°8 or SEQ ID N°9. More particularly fragments will retain some functional property, preferably an immunological activity, of the larger molecule set forth in SEQ ID N°5 or SEQ ID N°6 or SEQ ID N°7 or SEQ ID N°8 or SEQ ID N°9, and are useful in the methods described herein (e.g. in vaccine compositions, in diagnostics, etc.). In particular the fragments will be able to generate an immune response, when suitable attached to a carrier, that will recognise the protein of SEQ ID N°5 or SEQ ID N°6 or SEQ ID N°7 or SEQ ID N°8 or SEQ ID N°9.

Prostase homologues will generally share substantial sequence similarity, and include isolated polypeptides comprising an amino acid sequence which has at least 70% identity, preferably at least 80% identity, more preferably at least 90% identity, yet more preferably at least 95% identity, most preferably at least 97-99% identity, to that of SEQ ID N°5 or SEQ ID N°6 or SEQ ID N°7 or SEQ ID N°8 or SEQ ID N°9 over the entire length of SEQ ID N°5 or SEQ ID N°6 or SEQ ID N°7 or SEQ ID N°8 or SEQ ID N°9. Such polypeptides include those comprising the amino acid of SEQ ID N°5 or SEQ ID N°6 or SEQ ID N°7 or SEQ ID N°8 or SEQ ID N°9.

According to the present invention there is provided a process for purifying a prostase antigen, fragment or homologue thereof The process comprises treating the protein to reduce the protein's intra- and inter-molecular disulphide bonds, and blocking the thiol to prevent oxydative recoupling (referred to as “reduction/blocking step”). The reduction/blocking step may be carried out at the end of the purification process, on the purified antigen. Preferably however the reduction/blocking step is carried out during the purification process. Accordingly therefore there is provided a process comprising treating the protein to reduce the protein's intra- and inter-molecular disulphide bonds, blocking the thiol to prevent oxydative recoupling and subjecting the protein to one or more chromatographic steps. Advantageously, such steps lead to an increase of approximately 10 fold in protein yield, as without such steps, the protein aggregates and precipitates, reducing the effectiveness of downstream purification. Final purity and process consistency are also improved.

It is preferred to first solubilise the product in a strong chaotropic agent such as urea, guanidium hydrochloride. Zwitterionic detergents such as Empigen BB—n-dodecyl-N,N-dimethylglycine, or other detergents like for example Tween 80 (polyoxyethylene (20) sorbitan monooleate) may also be used. Optimally solubilisation involves the use of both a detergent and a chaotropic agent, optionally in the presence of the reducing agent. Preferably the solubilisation/reduction step is performed simultaneously, involving the use a detergent, a chaotropic agent and a reducing agent.

Prior to the alkylation of the protein it is preferred to filter the solubilise reduced protein, preferably through a 0.45 μm filter, such as a Vibrating Membrane Filtration (VMF).

Accordingly, in a preferred embodiment of the invention there is provided a method for the improved purification of the proteins of the present invention, wherein the protein is solubilised utilising a combination of both a strong chaotropic agent and a detergent. Preferably the detergent is in the range of 0.1% to 5%, more preferably from 0.2% to 2%, ideally from 0.5% to 1%, most preferably of 0.5%. Preferably the pH is in the range of 7 to 10, more preferably from 7.5 to 9.5, optimally between 7.5 and 9, ideally 8.5.

In another preferred embodiment the purification involves a first step of solubilising the protein preferably utilising both a strong chaotropic agent and a detergent, secondly, filtering the product, and subsequently reducing at least one, preferably substantially all, preferably all the protein's intra- and inter-molecular disulphide bonds utilising a reducing agent such as but not limited to glutathion, and blocking the free thiol groups, and subjecting the resulting protein to one or more chromatographic steps.

In another preferred embodiment the purification involves a first step of solubilisation/reduction using a strong chaotropic agent, a detergent and a reducing agent, secondly, filtering the product, thirdly blocking the free thiol groups, and then subjecting the resulting protein to one or more chromatographic steps. This provides up to 100-fold increase in yield of purified protein.

Preferably, the blocking agent is an alkylating agent. Such blocking agents include but are not limited to alpha haloacids or alpha haloamides. More preferably the alkylating agent is a carboxyalkylating agent. Still more preferably the carboxyalkylating agent is iodoacetic acid or iodoacetamide, which respectively results in carboxymethylation or carboxyamidation (carbamidomethylation) of the protein. Other blocking agents may be used and are described in the literature (See for example, The Proteins Vol II Eds H neurath, RL Hill and C-L Boeder, Academic press 1976, or Chemical Reagents' for Protein modification Vol I eds. RL Lundblad and CM Noyes, CRC Press 1985). Typical examples of such other blocking agents include N-ethylmaleimide, chloroacetyl phosphate, O-methylisourea and acrylonitrile. The use of the blocking agent is advantageous as it prevents the oxidation and subsequent aggregation of the prostase antigen and fusion derivatives, and ensure stability of the modified protein for downstream purification. The overall benefit is an increase in the purification yield, an enhanced product-purity as well as a consistency in the manufacture process.

One or more of these purification steps involves an ion-metal chelate affinity chromatography, preferably but not restricted to a Nickel-chelate affinity chromatography. These polypeptides can be purified to high levels (greater than 80% preferably greater than 90% pure as visualised by SDS-PAGE) by undergoing further purification steps. An additional purification step is a Q-Sepharose step that may be operated either before or after the IMAC column to yield highly purified protein. The proteins so purified present a major single band when analysed by SDS PAGE under reducing conditions, and western blot analysis show less than 10%, preferably less than 5% host cell protein contamination.

The reduction/blocking treatment also advantageously leads to the eliciting of antibody response at least as good, preferably better after injection of the modified protein as compared to the unmodified one.

In an embodiment of the invention the blocking agents are selected to induce a stable covalent and irreversible derivative (eg alpha halo acids or alpha haloamides).

However other blocking agents maybe selected such that after purification the blocking agent may be removed to release the non-derivatised protein. Prostase protein antigen, prostase fusion proteins and homologues, having derivatised free thiol residues are new and form an aspect of the invention. In particular alkylated derivatives are a preferred embodiment of the invention.

Carboxyamidated or carboxymethylated derivatives are a more preferred embodiment of the invention. Preferably the prostase protein according to the invention is selected from the group consisting of SEQ ID NO: 5, SEQ ID NO: 6, SEQ ID NO: 7, and SEQ ID NO: 8. Preferably the prostase fusion protein according to the invention has the sequence set forth in SEQ ID NO:3.

In a preferred embodiment of the invention the proteins of the present invention is provided with an affinity tag, such as a polyhistidine tail or a C-LYTA tag. In such cases the protein after the blocking step is preferably subjected to affinity chromatography. Preferably the affinity tag comprises a Histidine tail, fused at the carboxy-terminus of the proteins of the invention, preferably comprising between 5 to 8 histidine residues, preferably at least 4 residues, and most preferably 6 histidine residues. Preferably the affinity peptide has adjacent histidine residues, preferably at least two, more preferably at least 4 residues. Most preferably the protein comprises 6 directly neighbouring histidine residues. These histidine tag are designed in aiding the purification of the recombinant protein, particularly by Ni chelate based IMAC chromatography. In another preferred embodiment, the proteins are harbouring a C-LYTA tag at their carboxy-terminus. Preferably the C terminal portion of the molecule is used. Lyta is derived from Streptococcus pneumoniae which synthesize an N-acetyl-L-alanine amidase, amidase LYTA, (coded by the lytA gene {Gene, 43 (1986) page 265-272} an autolysin that specifically degrades certain bonds in the peptidoglycan backbone. The C-terminal domain of the LYTA protein is responsible for the affinity to the choline or to some choline analogues such as DEAE. This property has been exploited for the development of E.coli C-LYTA expressing plasmids useful for expression of fusion proteins. Purification of hybrid proteins containing the C-LYTA fragment at its amino terminus has been described {Biotechnology: 10, (1992) page 795-798}. As used herein a preferred embodiment utilises the repeat portion of the Lyta molecule found in the C terminal end starting at residue 178. A particularly preferred form incorporates residues 188-305. These preferential fusions are also new and form one aspect of the invention. For those proteins with a polyhistidine tail, immobilised metal ion affinity chromatography (IMAC) may be performed. The metal ion, may be any suitable ion for example zinc, nickel, iron, magnesium or copper, but is preferably zinc or nickel. Preferably the IMAC buffer contains detergent, preferably a non-ionic detergent such as Tween 80, or a zwitterionic detergent such as Empigen BB, as this may result in lower levels of endotoxin in the final product.

Further chromatographic steps include for example a Q-Sepharose step that may be operated either before of after the IMAC column. Preferably the pH is in the range of 7.5 to 10, more preferably from 7.5 to 9.5, optimally between 8 and 9, ideally 8.5.

The protein thus purified present a major single band when analysed by SDS-PAGE under reducing conditions and show less than 10%, preferably less than 5% host cell contamination as determined by Western blot analysis.

The proteins of the present invention are provided either soluble in a liquid form or in a lyophilised form, which is the preferred form. It is generally expected that each human dose will comprise 1 to 1000 μg of protein, and preferably 30-300 μg.

The prostase antigen derivative according to the invention or fragments and homologues thereof may in a preferred embodiment carry a mutation in the active site of the protein, to reduce substantially or preferably eliminate its protease biological activity. Preferred mutations involve replacing the Histidine and Aspartate catalytic residues of the serine protease. In a preferred embodiment, prostase contains a Histidine-Alanine mutation in the active site, for example at residue 71 of prostase (Ferguson, et al. (Proc. Natl. Acad. Sci. USA 1999, 96, 3114-3119) which corresponds to amino acid 43 of P703PDES sequence (depicted in SEQ ID N°8).

Accordingly, the chemically modified prostase whose sequence consists in SEQ ID NO:9 is a preferred embodiment of the invention. The mutation of the invention leads to a significant decrease in the catalytic efficiency (expressed in enzymatic specific activity) of the protein as compared to the non-mutated protein. Preferably the reduction in the catalytic efficiency is at least by a factor of 10³, more preferably at least by a factor of 10⁶. The protein which has undergone an histidine alanine mutation is hereafter referred to as * (star).

In another embodiment, the prostase antigen derivative according to the invention or fragments and homologues thereof are prostase fusion proteins, comprising the tumour-associated prostase or fragment or homologues thereof and a heterologous protein or-part of a protein acting as a fusion partner. The protein and the fusion partner may be chemically conjugated, but are preferably expressed as recombinant fusion proteins in a heterologous expression system. In a preferred embodiment of the invention there is provided a fusion protein comprising prostase derivatives according to the invention, or fragment or homologues thereof, linked to an immunological fusion partner that may assist in providing T helper epitopes. Thus the fusion partner may act through a bystander helper effect linked to secretion of activation signals by a large number of T cells specific to the foreign protein or peptide, thereby enhancing the induction of immunity to the prostase component as compared to the non-fused protein. Preferably the heterologous partner is selected to be recognizable by T cells in a majority of humans. In another embodiment, the invention provides a fusion protein comprising prostase derivatives according to the invention, or fragment or homologues thereof, linked to a fusion partner that acts as an expression enhancer. Thus the fusion partner may assist in aiding in the expression of prostase in a heterologous system, allowing increased levels to be produced in an expression system as compared to the native recombinant protein.

Preferably the fusion partner will be both an immunological fusion partner and an expression enhancer partner. Accordingly, the present invention in the embodiment provides fusion proteins comprising the tumour-specific prostase derivative or a fragment thereof linked to a fusion partner. Preferably the fusion partner is acting both as an immunological fusion partner and as an expression enhancer partner. Accordingly, in a preferred form of the invention, the fusion partner is the non-structural protein from influenzae virus, NS1 (hemagglutinin) or fragment thereof. Typically the N-terminal 81 amino acids are utilised, although different fragments may be used provided they include T-helper epitopes (C. Hackett, D. Horowitz, M. Wysocka & S. Dillon, 1992, J. Gen. Virology, 73, 1339-1343). When NS1 is the immunological fusion partner it has the additional advantage in that it allows higher expression yields to be achieved. In particular, such fusions are expressed at higher yields than the native recombinant prostase proteins.

In preferred embodiments, the prostase moiety within the fusion is selected from the group comprising SEQ ID NO:9 (mutated P703P), SEQ ID NO: 5 (Millenium WO 98/12302), SEQ ID NO: 6 (Incyte WO 98/20117), SEQ ID NO: 7 (PNAS (1999) 96, 3114-3119), and SEQ ID NO: 8 (Corixa WO 00/04149). Yet in a most preferred embodiment, the fusion protein comprises the N-terminal 81 amino acids of NS1 non structural protein fused to the 5 to 226 carboxy-terminal amino acids from mutated prostase, as set forth in SEQ ID NO: 1 (mutated prostase) and SEQ ID NO:3 (non mutated prostase).

The proteins of the present invention are expressed in an appropriate host cell, and preferably in yeast or in a bacterial host cell. More preferably the host cell is Pichia pastoris. Yet most preferably the host cell is E. coli. In a preferred embodiment the proteins are expressed with an affinity tag, such as for example, a histidine tail comprising between 5 to 9 and preferably six histidine residues, most preferably at least 4 histidine residues. These are advantageous in aiding purification through for example ion metal affinity chromatography (IMAC).

A DNA sequence encoding the proteins of the present invention can be synthesized using standard DNA synthesis techniques, such as by enzymatic ligation as described by D. M. Roberts et al. in Biochemistry 1985, 24, 5090-5098, by chemical synthesis, by in vitro enzymatic polymerization, or by PCR technology utilising for example a heat stable polymerase, or by a combination of these techniques. Enzymatic polymerisation of DNA may be carried out in vitro using a DNA polymerase such as DNA polymerase I (Klenow fragment) in an appropriate buffer containing the nucleoside triphosphates DATP, dCTP, dGTP and dTTP as required at a temperature of 10°-37° C., generally in a volume of 50 μl or less. Enzymatic ligation of DNA fragments may be carried out using a DNA ligase such as T4 DNA ligase in an appropriate buffer, such as 0.05M Tris (pH 7.4), 0.01 M MgCl₂, 0.01 M dithiothreitol, 1 mM spermidine, 1 mM ATP and 0.1 mg/ml bovine serum albumin, at a temperature of 4° C. to ambient, generally in a volume of 50 ml or less. The chemical synthesis of the DNA polymer or fragments may be carried out by conventional phosphotriester, phosphite or phosphoramidite chemistry, using solid phase techniques such as those described in ‘Chemical and Enzymatic Synthesis of Gene Fragments—A Laboratory Manual’ (ed. H. G. Gassen and A. Lang), Verlag Chemie, Weinheim (1982), or in other scientific publications, for example M. J. Gait, H. W. D. Matthes, M. Singh, B. S. Sproat, and R. C. Titmas, Nucleic Acids Research, 1982, 10, 6243; B. S. Sproat, and W. Bannwarth, Tetrahedron Letters, 1983, 24, 5771; M. D. Matteucci and M. H. Caruthers, Tetrahedron Letters, 1980, 21, 719; M. D. Matteucci and M. H. Caruthers, Journal of the American Chemical Society, 1981, 103, 3185; S. P. Adams et al., Journal of the American Chemical Society, 1983, 105, 661; N. D. Sinha, J. Biernat, J. McMannus, and H. Koester, Nucleic Acids Research, 1984, 12,4539; and H. W. D. Matthes et al., EMBO Journal, 1984, 3, 801.

In a further embodiment of the invention is provided a method of producing a protein as described herein. The process of the invention may be performed by conventional recombinant techniques such as described in Maniatis et al., Molecular Cloning—A Laboratory Manual; Cold Spring Harbor, 1982-1989.

In particular, the process of the invention may preferably comprise the steps of:

i) preparing a replicable or integrating expression vector capable, in a host cell, of expressing a DNA polymer comprising a nucleotide sequence that encodes the protein or an immunogenic derivative thereof;

ii) transforming a host cell with said vector;

ii) culturing said transformed host cell under conditions permitting expression of said DNA polymer to produce said protein; and

iv) recovering and purifying said protein, according to the method set out above.

The tern ‘transforming’ is used herein to mean the introduction of foreign DNA into a host cell. This can be achieved for example by transformation, transfection or infection with an appropriate plasmid or viral vector using e.g. conventional techniques as described in Genetic Engineering; Eds. S. M. Kingsman and A. J. Kingsman; Blackwell Scientific Publications; Oxford, England, 1988. The term ‘transformed’ or ‘transformant’ will hereafter apply to the resulting host cell containing and expressing the foreign gene of interest. Preferably recombinant antigens of the invention are expressed in unicellular hosts, most preferably in bacterial systems, most preferably in

E. coli.

Preferably the recombinant strategy includes cloning a gene construct encoding a NS1 fusion protein, the gene construct comprising from 5′ to 3′ a DNA sequence encoding NS1 joined to a DNA sequence encoding the protein of interest, into an expression vector to form a DNA fragment encoding a NS1-carboxyl-terminal P703P fusion protein. An affinity polyhistidine tail may be engineered at the carboxy-terminus of the fusion protein allowing for simplified purification through affinity chromatography.

The replicable expression vectors may be prepared by cleaving a vector compatible with the host cell to provide a linear DNA segment having an intact replicon, and combining said linear segment with one or more DNA molecules which, together with said linear segment encode the desired product, such as the DNA polymer encoding the protein of the invention, or derivative thereof, under ligating conditions.

Thus, the hybrid DNA may be pre-formed or formed during the construction of the vector, as desired.

The choice of vector will be determined in part by the host cell, which may be prokaryotic or eukaryotic but are preferably E. coli, yeast or CHO cells. Suitable vectors include plasmids, bacteriophages, cosmids and recombinant viruses. Expression and cloning vectors preferably contain a selectable marker such that only the host cells expressing the marker will survive under selective conditions. Selection genes include but are not limited to the one encoding protein that confer a resistance to ampicillin, tetracyclin or kanamycin. Expression vectors also contain control sequences which are compatible with the designated host. For example, expression control sequences for E. coli, and more generally for prokaryotes, include promoters and ribosome binding sites. Promoter sequences may be naturally occurring, such as the β-lactamase (penicillinase) (Weissman 1981, In Interferon 3 (ed. L. Gresser), lactose (lac) (Chang et al. Nature, 1977, 198: 1056) and tryptophan (trp) (Goeddel et al. Nucl. Acids Res. 1980, 8, 4057) and lambda-derived PL promoter system. In addition, synthetic promoters which do not occur in nature also function as bacterial promoters. This is the case for example for the tac synthetic hybrid promoter which is derived from sequences of the trp and lac promoters (De Boer et al., Proc. Natl Acad Sci. USA 1983, 80,21-26). These systems are particularly suitable with E. coli.

Yeast compatible vectors also carry markers that allow the selection of successful transformants by conferring prototrophy to auxotrophic mutants or resistance to heavy metals on wild-type strains. Control sequences for yeast vectors include promoters for glycolytic enzymes (Hess et al., J. Adv. Enzyme Reg. 1968, 7, 149), PHO5 gene encoding acid phosphatase, CUPI gene, ARG3 gene, GAL genes promoters and synthetic promoter sequences. Other control elements useful in yeast expression are terminators and leader sequences. The leader sequence is particularly useful since it typically encodes a signal peptide comprised of hydrophobic amino acids, which direct the secretion of the protein from the cell. Suitable signal sequences can be encoded by genes for secreted yeast proteins such as the yeast invertase gene and the a-factor gene, acid phosphatase, killer toxin, the a-mating factor gene and recently the heterologous inulinase signal sequence derived from INU1A gene of Kluyveromyces marxianus. Suitable vectors have been developed for expression in Pichia pastoris and Saccharomyces cerevisiae.

A variety of P. pastoris expression vectors are available based on various inducible or constitutive promoters (Cereghino and Cregg, FEMS Microbiol. Rev. 2000,24:45-66). For the production of cytosolic and secreted proteins, the most commonly used P. pastoris vectors contain the very strong and tightly regulated alcohol oxidase (AOX1) promoter. The vectors also contain the P. pastoris histidinol dehydrogenase (HIS4) gene for selection in his4 hosts. Secretion of foreign protein require the presence of a signal sequence and the S. cerevisiae prepro alpha mating factor leader sequence has been widly and successfully used in Pichia expression system. Expression vectors are integrated into the P. pastoris genome to maximize the stability of expression strains. As in S.cerevisiae, cleavage of a P. pastoris expression vector within a sequence shared by the host genome (AOX1 or HIS4) stimulates homologous recombination events that efficiently target integration of the vector to that genomic locus. In general, a recombinant strain that contains multiple integrated copies of an expression cassette can yield more heterologous protein than single-copy strain. The most effective way to obtain high copy number transformants requires the transformation of Pichia recipient strain by the sphaeroplast technique (Cregg et all 1985, Mol.Cell.Biol. 5: 3376-3385).

The preparation of the replicable expression vector may be carried out conventionally with appropriate enzymes for restriction, polymerisation and ligation of the DNA, by procedures described in, for example, Maniatis et al. cited above.

The recombinant host cell is prepared, in accordance with the invention, by transforming a host cell with a replicable expression vector of the invention under transforming conditions. Suitable transforming conditions are conventional and are described in, for example, Maniatis et al. cited above, or “DNA Cloning” Vol. II, D. M. Glover ed., IRL Press Ltd, 1985.

The choice of transforming conditions depends upon the choice of the host cell to be transformed. For example, in vivo transformation using a live viral vector as the transforming agent for the polynucleotides of the invention is described above. Bacterial transformation of a host such as E. coli may be done by direct uptake of the polynucleotides (which may be expression vectors containing the desired sequence) after the host has been treated with a solution of CaCl₂ (Cohen et al., Proc. Nat. Acad. Sci., 1973, 69, 2110), or with a solution comprising a mixture of rubidium chloride (RbCl), MnCl₂, potassium acetate and glycerol, and then with 3-[N-morpholino]-propane-sulphonic acid, RbCl and glycerol. Transformation of lower eukaryotic organisms such as yeast cells in culture by direct uptake may be carried out by using the method of Hinnen et al (Proc. Natl. Acad. Sci. 1978, 75: 1929-1933). Mammalian cells in culture may be transformed using the calcium phosphate co-precipitation of the vector DNA onto the cells (Graham & Van der Eb, Virology 1978, 52, 546). Other methods for introduction of polynucleotides into mammalian cells include dextran mediated transfection, polybrene mediated transfection, protoplast fusion, electroporation, encapsulation of the polynucleotide(s) into liposomes, and direct micro-injection of the polynucleotides into nuclei.

Culturing the transformed host cell under conditions permitting expression of the DNA polymer is carried out conventionally, as described in, for example, Maniatis et al. and “DNA Cloning” cited above. Thus preferably the cell is supplied with nutrient and cultured at a temperature below 50° C., preferably between 25° C. and 35° C., most preferably at 30° C. The incubation time may vary from a few minutes to a few hours, according to the proportion of the polypeptide in the bacterial cell, as assessed by SDS-PAGE or Western blot.

Where the host cell is bacterial, such as E. coli it may, for example, be lysed physically, chemically or enzymatically and the protein product isolated from the resulting lysate. Where the host cell is mammalian, the product may generally be isolated from the nutrient medium or from cell free extracts. Where the host cell is a yeast such as Saccharomyces cerevisiae or Pichia pastoris, the product may generally be isolated from from lysed cells or from the culture medium, and then further purified using conventional techniques. The specificity of the expression system may be assessed by western blot using an antibody directed against the polypeptide of interest.

The present invention also provides pharmaceutical composition comprising a protein of the present invention in a pharmaceutically acceptable excipient. A preferred vaccine composition comprises at least NS1-P703P*-His (SEQ ID N°1). Said protein has, preferably, blocked thiol groups and is highly purified, e.g. has less than 5% host cell contamination. Such vaccine may optionally contain one or more other tumour-associated antigen and derivatives. For example, suitable other associated antigen include PAP-1, PSA (prostate specific antigen), PSMA (prostate-specific membrane antigen), PSCA (Prostate Stem Cell Antigen), STEAP, and P501S (WO 98/37418).

Vaccine preparation is generally described in Vaccine Design (“The subunit and adjuvant approach” (eds. Powell M. F. & Newman M. J). (1995) Plenum Press New York). Encapsulation within liposomes is described by Fullerton, U.S. Pat. No. 4,235,877.

The proteins of the present invention are preferably adjuvanted in the vaccine formulation of the invention. Suitable adjuvants are commercially available such as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, and chemokines may also be used as adjuvants.

In the formulations of the invention it is preferred that the adjuvant composition induces an immune response predominantly of the TH1 type. High levels of Th1-type cytokines (e.g., IFN-γ, TNFα, IL-2 and IL-12) tend to favour the induction of cell mediated immune responses to an administered antigen. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173,1989.

Accordingly, suitable adjuvants for use in eliciting a predominantly Th1-type response include, for example a combination of monophosphoryl lipid A, preferably 3-de-O-acylated monophosphoryl lipid A (3D-MPL) together with an aluminium salt. Other known adjuvants, which preferentially induce a TH1 type immune response, include CpG containing oligonucleotides. The oligonucleotides are characterised in that the CpG dinucleotide is unmethylated. Such oligonucleotides are well known and are described in, for example WO 96/02555. Immunostimulatory DNA sequences are also described, for example, by Sato et al., Science 273:352, 1996. CpG-containing oligonucleotides may also be used alone or in combination with other adjuvants. For example, an enhanced system involves the combination of a CpG-containing oligonucleotide and a saponin derivative particularly the combination of CpG and QS21 as disclosed in WO 00/09159 and WO 00/62800. Preferably the formulation additionally comprises an oil in water emulsion and/or tocopherol.

Another preferred adjuvant is a saponin, preferably QS21 (Aquila Biopharmaceuticals Inc., Framingham, Mass.), that may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

A particularly potent adjuvant formulation involving QS21 3D-MPL & tocopherol in an oil in water emulsion is described in WO 95/17210 and is a preferred formulation.

Other preferred adjuvants include Montanide ISA 720 (Seppic, France), SAF (Chiron, Calif., United States), ISCOMS (CSL), MF-59 (Chiron), Detox (Ribi, Hamilton, Mont.), RC-529 (Corixa, Hamilton, Mont.) and other aminoalkyl glucosaminide 4-phosphates (AGPs).

Other preferred adjuvants include adjuvant molecules of the general formula (I):

HO(CH₂CH2O)ₙ-A-R

Wherein, n is 1-50, A is a bond or —C(O)—, R is C₁₋₅₀ alkyl or Phenyl C₁₋₅₀ alkyl.

One embodiment of the present invention consists of a vaccine formulation comprising a polyoxyethylene ether of general formula (I), wherein n is between 1 and 50, preferably 4-24, most preferably 9; the R component is C₁₋₅₀, preferably C₄-C₂₀ alkyl and most preferably C₁₂ alkyl, and A is a bond. The concentration of the polyoxyethylene ethers should be in the range 0.1-20%, preferably from 0.1-10%, and most preferably in the range 0.1-1%. Preferred polyoxyethylene ethers are selected from the following group: polyoxyethylene-9-lauryl ether, polyoxyethylene-9-steoryl ether, polyoxyethylene-8-steoryl ether, polyoxyethylene-4-lauryl ether, polyoxyethylene-35-lauryl ether, and polyoxyethylene-23-lauryl ether. Polyoxyethylene ethers such as polyoxyethylene lauryl ether are described in the Merck index (12^{th} edition: entry 7717). These adjuvant molecules are described in WO 99/52549.

The polyoxyethylene ether according to the general formula (I) above may, if desired, be combined with another adjuvant. For example, a preferred adjuvant combination is preferably with CpG as described in the pending UK patent application GB 9820956.2.

Accordingly in one embodiment of the present invention there is provided a vaccine comprising a chemically modified prostase, more preferably an alkylated prostase, still more preferably a carboxyalkylated prostase, most preferably a carboxyamidated or carboxymethylated prostase. A most preferred vaccine comprises to a carboxyamidated or carboxymethylated NS1-P703P*-His, adjuvanted with a one or more of CpG immunostimulatory oligonucleotide, monophosphoryl lipid A or derivative thereof, QS21 and tocopherol in an oil in water emulsion.

Preferably the vaccine comprises a saponin, more preferably QS21. Another particular suitable adjuvant formulation includes CpG and a saponin as described in WO 00/09159 and WO 00/62800 and is a preferred formulation. Most preferably the saponin in that particular formulation is QS21, preferably the less reactogenic form where QS21 is quenched with cholesterol, as described in WO 96/33739. Preferably the formulation additionally comprises an oil in water emulsion and tocopherol.

Any of a variety of delivery vehicles may be employed within pharmaceutical compositions and vaccines to facilitate production of an antigen-specific immune response that targets tumour cells. Delivery vehicles include antigen-presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumour effects per se and/or to be immunologically compatible with the receiver (i.e., matched HLA haplotype). APCs may generally be isolated from any of a variety of biological fluids and organs, including tumour and peri-tumoural tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumour immunity (see Timmerman and Levy, Ann Rev. Med. 50:507-529, 1999). In general, dendritic cells may be identified based on their typical shape (stellate in situ, with marked cytoplasmic processes (dendrites) visible in vitro), their ability to take up, process and present antigens with high. efficiency and their ability to activate naïve T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med 4:594-600, 1998).

Dendritic cells and progenitors may be obtained from peripheral blood, bone marrow, tumour-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFA, CD40 ligand, lipopolysaccharide LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide encoding prostase tumour protein (or derivative thereof) such that the prostase tumour polypeptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the prostase tumour polypeptide, DNA (naked or within a plasmid vector) or RNA; or with antigen-expressing recombinant bacterium or viruses (e.g., vaccinia, fowlpox, adenovirus or lentivirus vectors).

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

The present invention also provides a process for the production of a vaccine, comprising the steps of purifying a prostase protein or a derivative thereof, by the process disclosed herein and admixing the resulting protein with a suitable adjuvant, diluent or other pharmaceutically acceptable excipient.

The present invention also provides a method for producing a vaccine formulation comprising mixing a protein of the present invention together with a pharmaceutically acceptable excipient, such as 3D-MPL.

Another aspect of the invention is the use of a protein as claimed herein for the manufacture of a vaccine for immunotherapeutically treating a patient suffering from prostate cancer or other prostase-associated tumours. A method of treating patients susceptible to or suffering from prostate-cancer comprising administering to said patients a pharmaceutically active amount of the vaccine disclosed herein is also contemplated by the present invention.

### FIGURES LEGENDS

FIG. 1: Design of the fusion protein NS 1-p703*-His expressed in E. coli

FIG. 2: Primary structure of the fusion protein NS 1-p703 *-His expressed in E. coli (SEQ ID N°1).

FIG. 3: Coding sequence of NS₁₋₈₁-P703P*-His (SEQ ID N°2).

FIG. 4: Cloning strategy to produce NS1-P703P*-His in

E. coli

FIG. 5: Plasmid map of RIT 14952

FIG. 6: E. coli NS 1-P703P*-His fermentation process

FIG. 7: E. coli NS1-P703P*-His purification process

FIG. 8: Characterisation of NS1-P703P*-His. The reducing buffer (SB+) contains (final concentration) 7.25% v/v glycerol, 1.45% w/v SDS, 0.5 M 2-mercaptoethanol, 0.0033% w/v bromophenol blue and 58 mM Tris buffer pH 6.8. The non-reducing buffer (SB−) does not contain 2-mercaptoethanol. P2F stands for VMF permeate containing the antigen. P2F R/C stands for VMF containing the reduced/carboxyamidated antigen. FT stands for IMAC flow through (lane 9) and W stands for IMAC wash (lane 10).

FIG. 9: Immunogenicity of NS1-P703P*-His adjuvanted with SBAS2

FIG. 10: Primary structure of the fusion protein NS1-p703-His expressed in E. coli (SEQ ID N°3).

FIG. 11: Coding sequence of NS₁₋₈₁-P703P-His (SEQ ID N°4).

FIG. 12: Primary structure of the protein p703*-His expressed in Pichia pastoris (SEQ ID N°9).

FIG. 13: Coding sequence of P703P*-His expressed in Pichia pastoris (SEQ ID N°10).

FIG. 14: Plasmid map of pRIT 15043

The invention will be further described by reference to the following examples:

### EXAMPLE I

Preparation of the Recombinant E. Coli Strain Expressing the Fusion Protein NS1-P703P*-3-His

1.—Protein Design

The expression strategy followed for this candidate included the design of the most appropriate primary structure for the recombinant protein that could have the best expectation for both, good level of expression and easy purification process.

Although the chance that a recombinant protein could keep its protease biological activity when formulated for vaccination is really very low, the mutation of the active side was made in order to reduce substantially or preferably eliminate its proteolytical biological activity. Accordingly, the His residue at position 43 of SEQ ID N°8, has been mutated into an Ala residue.

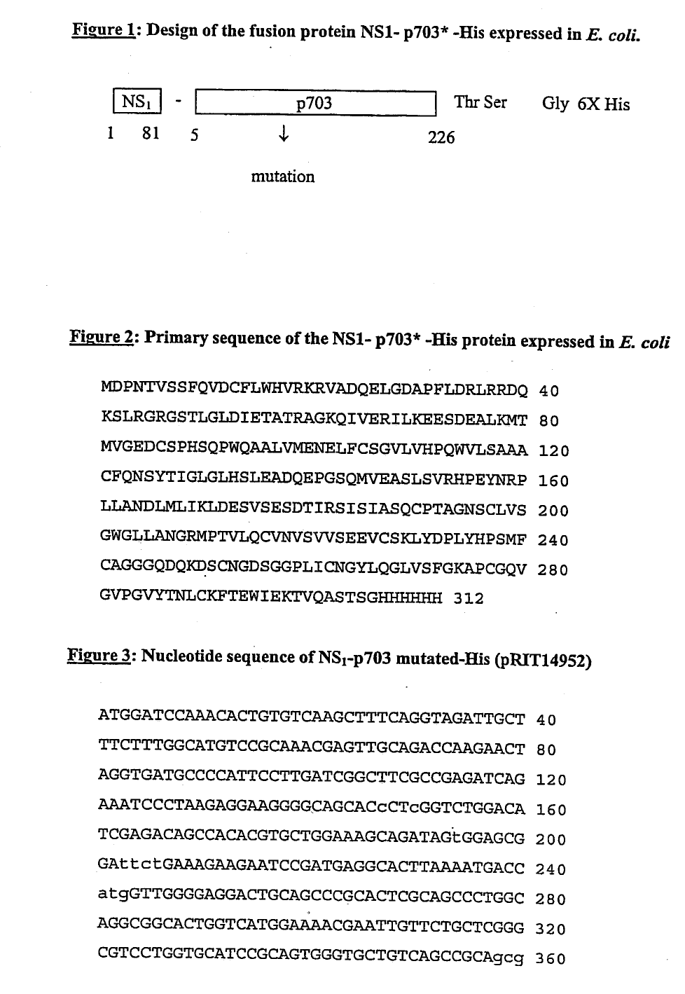

The design of the fusion protein NS1-p703*-His to be expressed in E. coli is described in FIG. 1. This fusion contains the N-terminal (81 amino acid) of non structural protein of Influenzae virus, followed by the non processed amino acid sequence of prostate antigen (amino acids 5→226 of p703pde5 sequence described in SEQ ID N°8 containing the mutation His→Ala of the 43 residue of the protease active site followed by the His tail. The Histidine tail was added to prostase to enable versatile purification of the fusion and processed protein. The length of the fusion is 313 aminoacids.

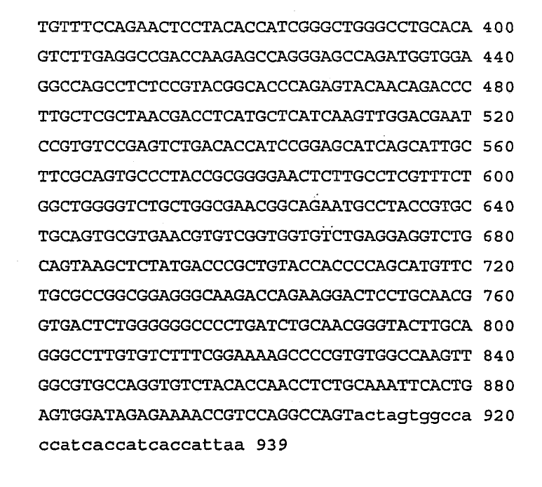

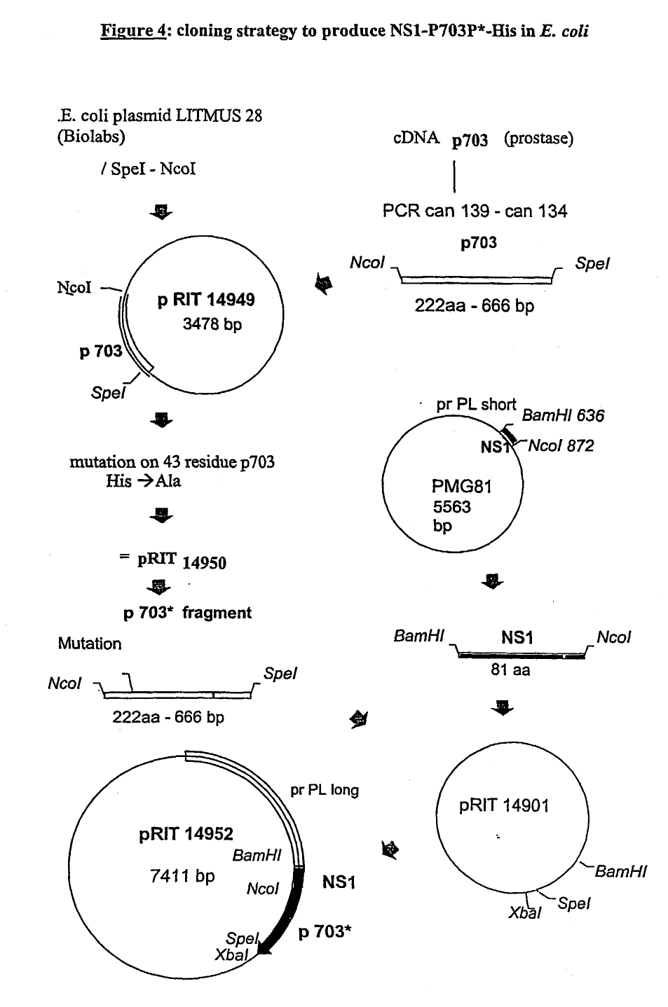

The primary structure of the resulting protein has the sequence described in FIG. 2. The coding sequence corresponding to the above protein is illustrated in FIG. 3 and was subsequently placed under the control of λpL promoter in a E. coli expression plasmid.

2.—The E. Coli Expression System

For the production of NS1 the DNA encoding the 81 amino-terminal residues of NS1 (non-structural protein from influenzae virus) has been cloned into the expression vector pMG 81. This plasmid utilises signals from lambda phage DNA to drive the transcription and translation of inserted foreign genes. The vector contains the lambda PL promoter PL, operator OL and two utilisation sites (NutL and NutR) to relieve transcriptional polarity effects when N protein is provided (Gross et al., 1985. Mol. & Cell. Biol. 5:1015). Vectors containing the PL promoter, are introduced into an E. coli lysogenic host to stabilise the plasmid DNA. Lysogenic host strains contain replication-defective lambda phage DNA integrated into the genome (Shatzman et al., 1983; In Experimental Manipulation of Gene Expression. Inouya (ed) pp 1-14. Academic Press NY). The lambda phage DNA directs the synthesis of the cI repressor protein which binds to the OL repressor of the vector and prevents binding of RNA polymerase to the PL promoter and thereby transcription of the inserted gene. The ci gene of the expression strain AR58 contains a temperature sensitive mutation so that PL directed transcription can be regulated by temperature shift, i.e. an increase in culture temperature inactivates the repressor and synthesis of the foreign protein is initiated. This expression system allows controlled synthesis of foreign proteins especially of those that may be toxic to the cell (Shimataka & Rosenberg, 1981. Nature 292:128).

3.—The E. Coli Strain AR58:

The AR58 lysogenic E. coli strain used for the production of the NS1-P703P*-His protein is a derivative of the standard NIH E.coli K12 strain N99 (F-su-galK2, lacZ-thr-). It contains a defective lysogenic lambda phage (galE::TN10, 1 Kil-cI857 DH1). The Kil-phenotype prevents the shut off of host macromolecular synthesis. The cI857 mutation confers a temperature sensitive lesion to the cI repressor. The DH1 deletion removes the lambda phage right operon and the hosts bio, uvr3, and chlA loci. The AR58 strain was generated by transduction of N99 with a P lambda phage stock previously grown on an SA500 derivative (galE::TN10, 1 Kil-cI857 DH1). The introduction of the defective lysogen into N99 was selected with tetracycline by virtue of the presence of a TN10 transposon coding for tetracyclin resistance in the adjacent galE gene. N99 and SA500 are E.coli K12 strains derived from Dr. Martin Rosenberg's laboratory at the National Institutes of Health.

4.—Construction of the Vector Designed to Express the Recombinant Protein NS1-P703P*-His

The starting materials were:

1) A cDNA plasmid received from CORIXA p703pde5 (WO 00/04149), where the putative signal sequence and a piece of the pro-peptide of P703P is missing (see FIG. 1) and containing the coding sequence for prostase antigen;

2) Vector pRIT14901 containing the long version of the PL promoter; and

3) Plasmid PMG81 containing the 81aa of NS₁ coding region from Influenzae virus.

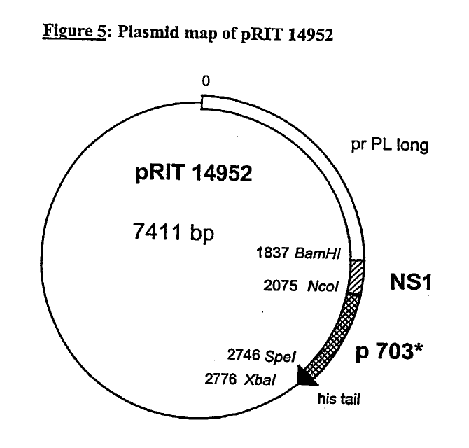

The cloning strategy outlined in FIG. 4 included the following steps:

a) PCR amplification of the p703 sequence with NcoI and SpeI restriction sites The template for the PCR reaction was the cDNA plasmid received from CORIXA, the oligonucleotide sense can139: 5′GCG CCC ATG GTT GGG GAG GAC TGC AGC CCG 3′, and the oligonucleotide antisense can134 5′GGG ACT AGT ACT GGC CTG GAC GGT TTT CTC 3′;

b) Insertion of the amplified sequences into the commercial vector Litmus 28 (biolabs), leading to the intermediate plasmid pRIT 14949;

c) Directed His→Ala mutagenesis of residue 43 of the p703 sequence contained in the plasmid pRIT 14949, using the sense oligonucleotide can140: 5′CTG TCA GCC GCA GCG TGT TTC CAG 3′and the antisense oligonucleotide can141:5′CTG GAA ACA CGC TGC GGC TGA CAG 3′, leading to the obtention of plasmid pRIT 14950;

d) Isolation of the NcoI-SpeI fragment from the plasmid pRIT 14950;

e) From pMG81 plasmid, purification of NS1 fragment (81aa) after digestion of the restriction sites BamHI-NcoI;

f) Ligation of both fragments were ligated to the expression plasmid pRIT 14901 (pr PL long);

g) Selection and characterisation of the E. coli AR58 strain transformants containing the plasmid pRIT14952 (see FIG. 5) expressing the NS1—p703 mutated—His fusion protein

The recombinant strain thus produces the NS1-P703P* His-tailed fusion protein of 313 amino acid residues long (see FIG. 2), with the amino acids sequence described in ID NO:1 and the coding sequence is described in ID NO:2.

### EXAMPLE II

Preparation of the Recombinant NS1-P703P*-3-His Fusion Protein

1.—Growth and Induction of Bacterial Strain B1225—Expression of NS1-P703P*-3-His

Cells of AR58 transformed with plasmid pRIT14952 (strain B1225) were grown in a 2 L flask containing 400 ml FECO15AA medium supplemented with kanamycin sulphate (100 mg/L). After a 16h of incubation at 30° C. and at 200 rpm, a small sample was removed from this flask for microscopic examination. 50 ml of this pre-culture was transferred into a 20-L fermentor containing 8.7 L of FEC012AF medium supplemented with kanamycin sulphate (50 mg/L). The pH was adjusted to and maintained at 6.8 by addition of NH4OH (25% v/v), and the temperature was maintained at 30° C. The aeration rate was kept constant at 20 L/min and the pO2 was regulated at 20% of saturation by feedback control of the agitation speed. The head pressure was maintained at 0.5 bar.

This fed-batch fermentation process is based on glycerol as a carbon source. The feed solution was added at an initial rate of 0.04 ml/min, and increased exponentially during the first 30 hours to limit the growth rate in order to be able to keep a minimum pO2 level of 20%.

After 30 hours, the temperature of the fermentor was rapidly increased to 39.5° C. in order to induce the intracellular expression of the antigen NS1-P703P*-His. The feeding rate was maintained constant at 1.28 ml/min during the whole induction phase (18h). Samples of broth were taken during both growth and induction phases in order to monitor bacterial growth and antigen expression. Microbiological identification and purity tests were also realised on these materials.

At the end of fermentation, the biomass reached an optical density of about 130, corresponding to a dry cell weight of about 50g/L. The final volume was approximately 10.5 L. The cells containing the antigen were directly separated from the culture medium by centrifugation at 5000 g for 1 h at 4° C. and the pellet was stored in plastic bags at −70° C.

2.—Extraction of the Protein:

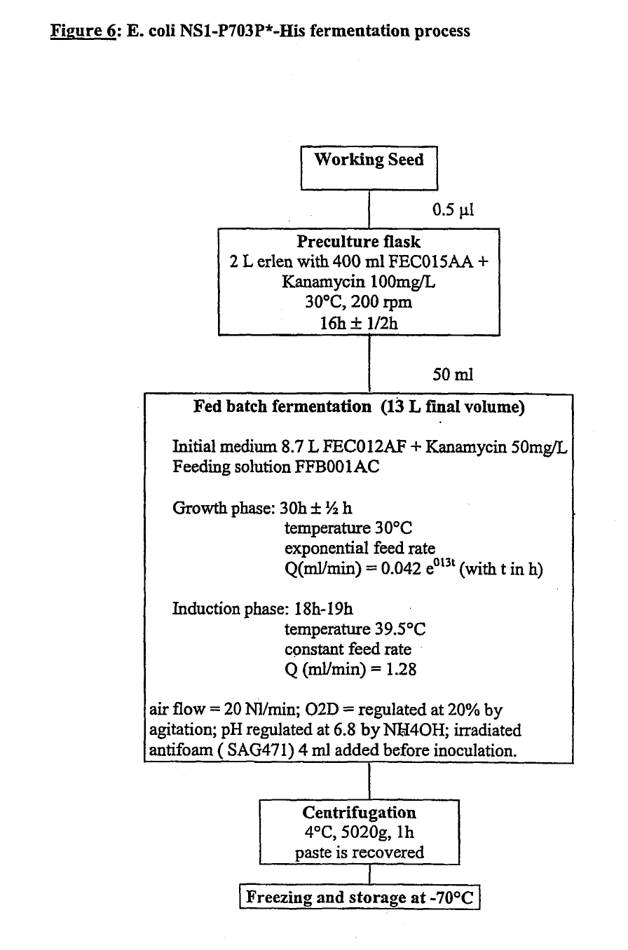

Recombinant NS1-P703P*-His protein, expressed in E. coli as inclusion bodies, was purified from cell homogenate using different steps (see FIG. 6). Briefly, frozen concentrated cells from fermentation harvest were thawed to +4° C. before being resuspended in disruption buffer (phosphate 20 mM—NaCl 2M—EDTA 5 mM pH 7.5) to a final optical density (OD650) of 120. Two passes through a high-pressure homogeniser (1000 bars) disrupted the cells.

### EXAMPLE III

Characterisation of Fusion Protein NS1-P703P*-His

1.—Purification Process:

a) Introduction

As said above, the recombinant protein, NS1-P703P*-His is produced in E. coli in the form of inclusion bodies. A major issue for the set-up of the purification method was the oxidation of the recombinant protein with itself or with host cell contaminants, likely through covalent binding with disulphide bonds. The process as developed aimed at reducing the massive oxidation phenomenon in order to have a highly purified product together with an acceptable global yield, while preserving the product ability to mount an effective immune response against the antigen of interest. It has been used to produce GMP material.

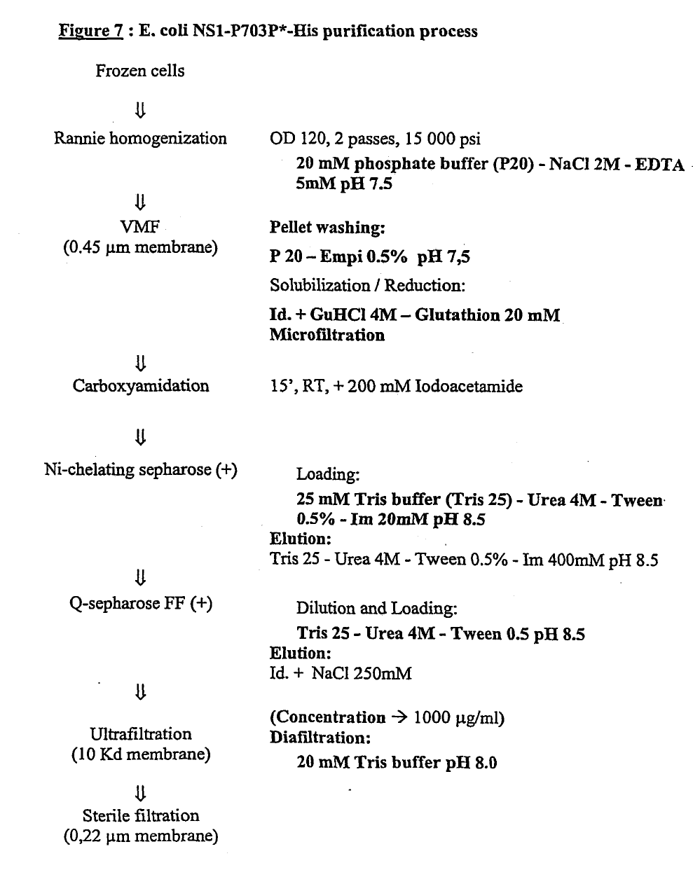

b) Description of the Process (FIG. 7)

The broken cell suspension was treated on a Pallsep VMF (Vibrating Membrane Filtration) system (Pall-Filtron) equipped with 0.45 μm membrane. The “pellet fraction” was first washed by diafiltration with 20 mM phosphate buffer pH 7.5 containing 0.5% Empigen BB detergent. The washed material was then solubilised in the same buffer containing 4M guanidine hydrochoride and 20 mM glutathion. The product was recovered through 0.45 μm filter and the permeate was treated with 200 mM iodoacetamide to prevent oxidative re-coupling.

The carboxyamidated fraction was subjected to IMAC (Nickel-Chelating-Sepharose FF, Pharmacia). The column was first equilibrated with 25 mM Tris buffer pH 78.5 containing 4M urea, 0.5% (v:v) Tween 80, and 20 mM imidazole. After the sample loading, the column was washed with the same buffer. The protein was then eluted in the previous buffer with 400 mM Imidazole.

Before continuing the anion exchange chromatography, the conductance of the IMAC-eluate was reduced to below 5 mS/cm (3.5 mS/cm) with 25 mM Tris buffer pH 8.5 containing 4M urea and 0.5% (v:v) Tween 80. The packed bed support (Q-Sepharose FF, Pharmacia) was equilibrated with the dilution buffer. After the sample loading and a washing step with the equilibration buffer, the protein was eluted with the same buffer containing 250 mM NaCl.

The Q-Sepharose FF-eluate was then diafiltered against the appropriate storage buffer (25 mM Tris buffer pH 8.0) in a tangential flow filtration unit equipped with a 10 Kd cut-off membrane (Omega, Filton). Ultrafiltration retentate containing NS1-P703P*-His was sterile filtered through 0.22 μm membrane.

The global purification yield was very high: between 2-4 g (2.5 g on average) of purified material/L of homogenate (DO0120).

2.—Characterisation of the Purified Protein:

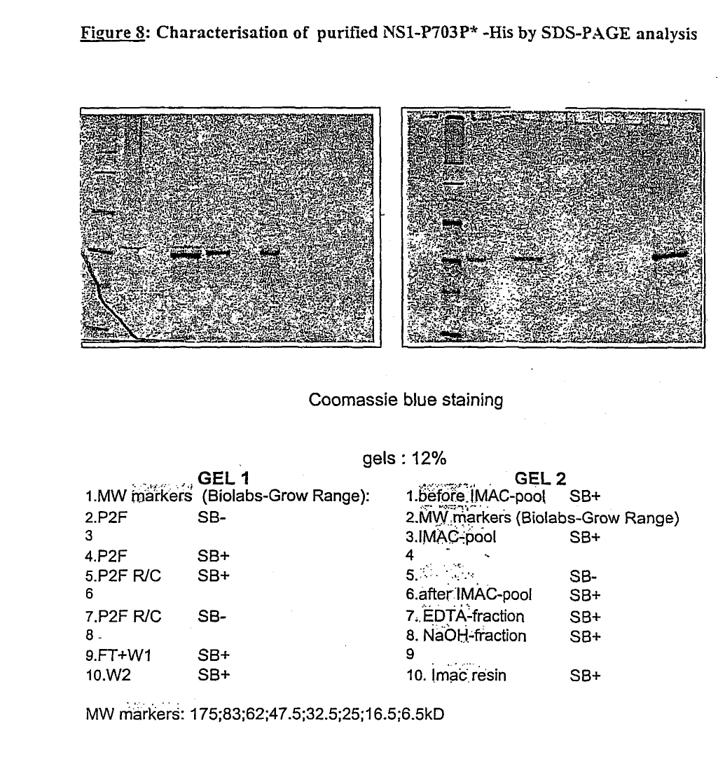

Follow-up of the purification of NS1-P703P*-His antigen was analysed by SDS-PAGE on a 12% acrylamide gel both in reducing and in non-reducing conditions (FIG. 8). According to SDS-PAGE analysis (Silver Staining/Coomassie/Western Blotting), the recombinant protein as purified by the optimised process, was composed of 1 major band at the expected MW and 2 or 3 minor bands of lower MW. Taking into account all these bands, purity was estimated ≧95%. The introduction of a solubilisation step using urea and a detergent allowed to improve the global purification yield by roughly a 10-fold increase: up to 300 mg of purified material/L of fermentation broth was obtained, with one major band on SDS-PAGE. An additional 10-fold increase of the process yield was obtained by the introduction of the reduction/carboxyamidation step. Purity of the final product with regards to host cell contaminants was also improved, and further oxidation of the product was avoided as evidenced by a similar pattern between the reduced and non-reduced material (FIG. 8, gel 1, lane 7 as compared to lane 5 respectively). FIG. 8, gel 1, lanes 9 and 10 show that the carboxyamidated protein when treated with a combination of 0.5% Tween 80 detergent and 4M urea chaotropic agent, shows a correct binding to the IMAC column, since no protein was recovered in the flow-through (lane 9) nor in the washings (lane 10). FIG. 8, gel 2, shows patterns of the IMAC eluate in reducing (lane 3) and non-reducing (lane 5) buffer, which is a further confirmation of the stabilisation of the oxidation. No protein of interest is detected in the consecutive elution fractions (gel 2, lanes 6-8). Other residual contaminants (endotoxin, DNA) were below the usual specification limits: respectively, 30 EU and 100 ng/100 μg protein.

According to SDS-PAGE analysis, the purified material was stable at +4° C. and +37° C. (1 week) although some precipitation was observed after freeze-thawing cycles. This precipitation can be avoided if the freezing is achieved in the presence of sucrose.

3.—Conclusions:

The purification process developed stepwise has allowed enhancing by a 100-fold factor the production yield of the fusion protein. The final product that is being recovered harbours much lower contamination by host cell proteins, lower oxidised pattern, lower aggregation, higher stability and much more consistency from batch to batch, all features that are compatible with high-scale production for industrial applications.

### EXAMPLE IV

Vaccine Preparation Using NS1-P703P*-His Protein

1.—Vaccine Preparation Using NS1-P703P*-His Protein:

The vaccine used in these experiments is produced from a recombinant DNA, encoding a NS1-P703P*-His, expressed in E. coil from the strain AR58, either adjuvanted or not. As an adjuvant, the formulation comprises a mixture of 3 de —O— acylated monophosphoryl lipid A (3D-MPL) and QS21 in an oil/water emulsion. The adjuvant system SBAS2 has been previously described WO 95/17210.

3D-MPL: is an immunostimulant derived from the lipopolysaccharide (LPS) of the Gram-negative bacterium Salmonella Minnesota. MPL has been deacylated and is lacking a phosphate group on the lipid A moiety. This chemical treatment dramatically reduces toxicity while preserving the immunostimulant properties (Ribi, 1986). Ribi Immunochemistry produces and supplies MPL to SB-Biologicals. Experiments performed at Smith Kline Beecham Biologicals have shown that 3D-MPL combined with various vehicles strongly enhances both the humoral and a TH1 type of cellular immunity.

QS21: is a natural saponin molecule extracted from the bark of the South American tree Quillaja saponaria Molina. A purification technique developed to separate the individual saponines from the crude extracts of the bark, permitted the isolation of the particular saponin, QS21, which is a triterpene glycoside demonstrating stronger adjuvant activity and lower toxicity as compared with the parent component. QS21 has been shown to activate MHC class I restricted CTLs to several subunit Ags, as well as to stimulate Ag specific lymphocytic proliferation (Kensil, 1992). Aquila (formally Cambridge Biotech Corporation) produces and supplies QS21 to SB-Biologicals.

Experiments performed at SmithKline Beecham Biologicals have demonstrated a clear synergistic effect of combinations of MPL and QS21 in the induction of both humoral and TH1 type cellular immune responses.

The oil/water emulsion is composed an organic phase made of of 2 oils (a tocopherol and squalene), and an aqueous phase of PBS containing Tween 80 as emulsifier. The emulsion comprised 5% squalene 5% tocopherol 0.4% Tween 80 and had an average particle size of 180 nm and is known as SB62 (see WO 95/17210).

Experiments performed at SmithKline Beecham Biologicals have proven that the adjunction of this O/W emulsion to 3D-MPL/QS21 (SBAS2) further increases the immunostimulant properties of the latter against various subunit antigens.

2.—Preparation of Emulsion SB62 (2 Fold Concentrate):

Tween 80 is dissolved in phosphate buffered saline (PBS) to give a 2% solution in the PBS. To provide 100 ml two fold concentrate emulsion 5 g of DL alpha tocopherol and 5 ml of squalene are vortexed to mix thoroughly. 90 ml of PBS/Tween solution is added and mixed thoroughly. The resulting emulsion is then passed through a syringe and finally microfluidised by using an M11OS microfluidics machine. The resulting oil droplets have a size of approximately 180 nm.

3.—Lyophilisation of NS1-P703P*-His:

In practice, all compounds are placed in solution and sterilisation is achieved by filtration on a 0.2 μm membrane. Formulations were performed the day of freeze-drying.

The sequence of formulation was:

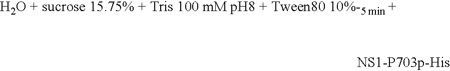

The volumes of all compounds are adjusted to have in final:

250-50-10 μg NS1-P703P*-His, in Tris 10 mM, tween 8O 0.2%, 3.15% sucrose.

The vial was overfilled with by 1.25×(reconstitution with 625 μl diluant, injection of 500 μl).

Using the Lyovac GT6 lyophilisation apparatus purchased from Steris (Germany), the lyophilisation cycle was performed during 3 days as follows:

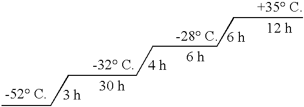

4.—Preparation of NS1-P703P*-His QS21/3D MPL Oil in Water (AS02) formulation:

The adjuvant is formulated as a combination of MPL and QS21, in an oil/water emulsion.

1) Formulation composition (injection volume: 100 μl); group 1 has received P703P*-His (20 μg) formulated in a combination of MPL and QS21, in an oil/water emulsion. Group 2 has received NS1-P703P*-His (25 μg) in a combination of MPL and QS21, in an oil/water emulsion.

2) Components

| Components | Conc mg/ml | Buffer |
|---|---|---|
| P703p-His (Pichia) | 0.513 | Po4 20 mM pH 7.5 |
| NS1-P703p-his carboxy | 0.846 | Tris 20 mM Tween 80 0.2% |
| | | pH 7.5 |
| SB62 | 2 x | PBS pH 6.8 |
| MPL | 8.175 | H₂O |
| QS21 | 2 | H₂O |
| Thiomersal | 0.2 | H₂O |

3) Formulations

The formulations were prepared extemporaneously on the day of injection.

The formulations containing 3D-MPL and QS21 in an oil/water emulsion (AS02B formulations—Groups 2 and 3) were performed as follows: P703p (20 μg) (group 2) and NS1-P703P*-His (25 μg) (group 3) were diluted in 10-fold concentrated PBS pH 6.8 and H₂O before consecutive addition of SB62 (50 μl), MPL (20 μg), QS21 (20 μg) and 1 μg/ml thiomersal as preservative at 5 min intervals. All incubations were carried out at room temperature with agitation.

The non-adjuvanted formulations (Groups 4 and 5) were performed as follows: P703p (20 μg) (group 4) and NS1-P703P*-His (25 μg) (group 5) were diluted in 1.5 M NaCl and H₂O before addition of 1 μg/ml thiomersal as preservative at 5 min intervals. All incubations were carried out at room temperature with agitation.

The final vaccine is obtained after reconstitution of the lyophilised NS1-P703P*-His preparation with the adjuvant or with PBS alone.

The adjuvant controls without antigen were prepared by replacing the protein by PBS.

### EXAMPLE V

Immunogenicity Using NS1-P703P*-His Protein

1.—Immunogenicity of NS1-P703P*-His in Mice. First Experiment

The aim of the experiment was to characterise the immune response induced in mice by vaccination with the purified recombinant mutated NS1-p703*-His molecule produced in E coli, in the presence or the absence of an adjuvant.

a)—Immunization Protocol:

Groups of 10 immunocompetent Balb/c mice, 6 to 8 weeks old mice, were vaccinated twice, intramuscularly, at 2 weeks interval with 25 μg of mutated NS1-P703-His formulated or not in AS02B (501 μl SB62/10 μg MPL/10 μg QS21). 14 days after the second injection, blood was taken and the sera were tested for the presence of anti-P703 antibodies.

b)—Total IgG Antibody Response:

The anti P703 antibody response has been assessed in the sera of the mice 14 days after the latest vaccination. This has been done by ELISA using either carboxyamidated or non carboxyamidated NS1-P703P*-His as coating antigen.

E coli extracts were used to check for the possible presence of antibodies against host contaminants.

c)—Results:

The results show that 1) a higher immune response (IgG) is induced by NS1-P703P*-His as evidenced in the sera of mice injected with the carboxyamidated NS1-P703P*-His protein alone as compared to the sera of normal control mice; 2) high antibody titers are found in animals receiving the NS1-P703P*-His carboxyamidated molecule formulated in the AS02B adjuvant; and 3) the antibodies recognise both the carboxyamidated (NS1-P703P*-His) and non carboxyamidated native form of the molecule (NS1-P703P*-HisNC).

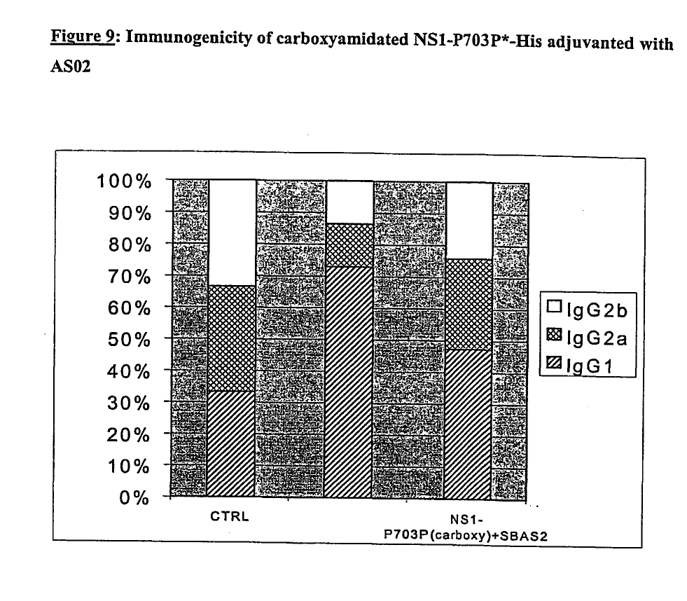

The isotypic profile of the NS 1-p703p-His specific IgG response has also been measured. As shown in FIG. 9, IgG1 were detected when mice received the carboxyamidated NS1-P703P*-His protein alone, however the isotypic profile was pushed towards a TH1 response (more IgG2a) by the presence of the AS02 adjuvant.

2.—Immunogenicity of NS1-P703P*-His in Mice. Second Experiment (LAS20000703)

The aim of the experiment was to determine whether antibodies generated in mice by vaccination with the carboxyamidated NS1-p703*-His molecule produced in E coli were cross-reacting with non carboxyamidated and carboxyamidated Pichia-produced P703P*-His (see Example VIII). NS1-OspA is an unrelated antigen.

a)—Immunization Protocol:

Groups of 8-weeks old DBA2 (n—10) mice received intramuscular injection at days 0-14-2842 of either PBS buffer or 25 μg of mutated carboxyamidated NS1-P703*-His C formulated or not in AS02B (25 μl SB62/10 μg MPL/10 μg QS21) or AS01B. AS01B is prepared by adding QS21 (5 μg) to small unilamellar vesicles (SUV) of dioleoyl phosphatidylcholine containing cholesterol (25 μg) (WO 96/33739) and MPL (5 μg) in the membrane. Half of the mice were sacrificed after 2 injections and the other half after 4 vaccinations in order to assess the serology.

b)—Total IgG Antibody Response:

The anti P703 antibody response has been assessed in the sera of the mice 14 days after the latest vaccination. This has been done by ELISA using either Pichia-produced carboxyamidated P703P*-His C, Pichia-produced non-carboxyamidated P703P*-His NC and E coli produced carboxyamidated NS1-P703P*-His C antigen, as coating antigen. The results obtained post II are shown in Table 1. They have been confirmed post IV.

TABLE 1
(14 post II)

| | | | NS1- | NS1- |
|---|---|---|---|---|
| | | NS1- | P703P*- | P703P*- |
| | PBS- | P703P*- | His | His |
| Coatings | Control | His | AS02B | AS01B |
| NS1-P703P-His | 50 | 607 | 76018 | 89340 |
| Pichia P703P NC | 50 | 109 | 8205 | 10167 |
| Pichia P703P C | 50 | 175 | 37110 | 27172 |
| NS1-OspA | 50 | 50 | 13293 | 11945 |

c)—Results:

The results show that 1) the antibody response seen in the P703P vaccinated mice was higher than that seen in the serum of the control mice vaccinated with PBS buffer; 2) a higher immune response (total IgG) is induced by NS1-P703P*-His as compared to the response seen adjuvanted with AS02B and AS01B as compared to the non-adjuvanted protein; 3) the response against the carboxyamidated Pichia-produced P703P*-His was more important than the response against the non carboxyamidated Pichia-produced P703P*-His. This may suggests that certain B cell epitopes were generated by the carboxyamidation process.

### EXAMPLE VI

Preparation of the Recombinant E.Coli Strain Expressing the Unmutated NS1-P703P-His

1.—NS1-P703P-His

In an analogous fashion NS1-P703P-His was prepared. The amino acid and DNA sequences are depicted in SEQUENCE ID Nos. 3 and 4.

Briefly, the strategy to express a NS1-P703P-His fusion protein in E.coli included the following steps:

a) As a starting materiel, the same starting material as described in Example I (amino acids 5→226 of p703pde5 sequence described in SEQ ID N°8);

b) PCR amplification to flank the p703 unmutated sequence cloning restriction sites;

c) Insertion in a PMG81 vector (promoter pL long) containing the NS1 gene;

d) Transformation of the recipient strain AR58 or AR120

e) Selection of recombinant strain.

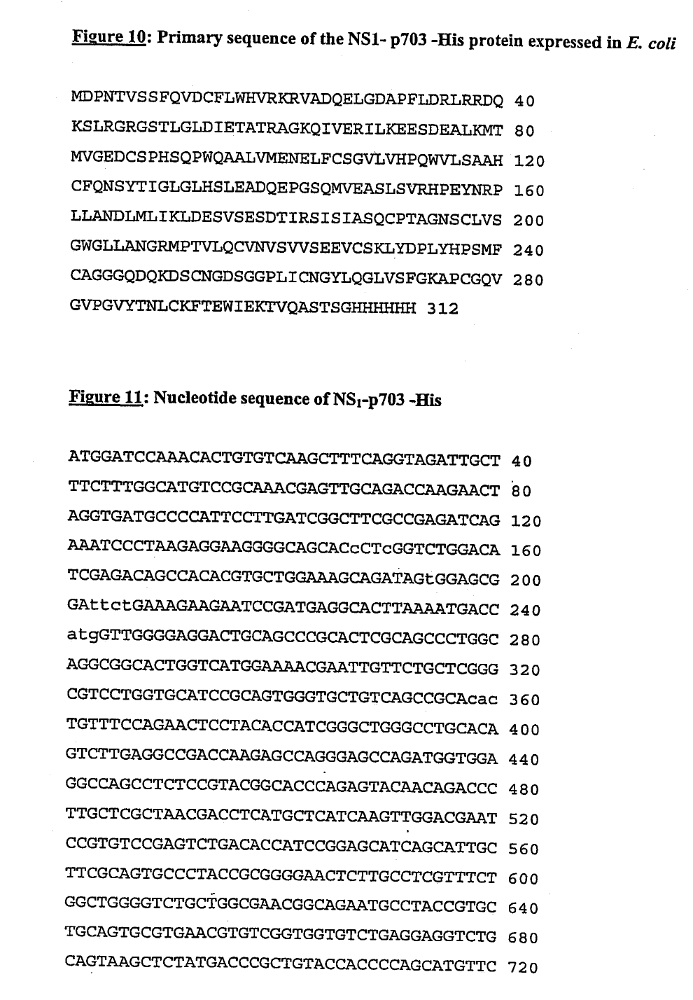

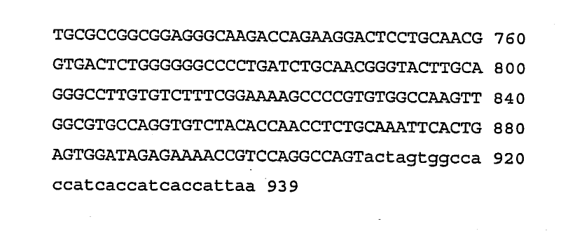

The resulting protein can be purified in an analogous manner to the NS1-P703P*-His mutated protein. The primary structure of the resulting protein has the sequence described in FIG. 10. The coding sequence corresponding to the above protein is illustrated in FIG. 11.

### EXAMPLE VII

Preparation of the Recombinant Pichia pastoris Strain Expressing the Protein P703P*-His

1.—Protein Design

Mutated P703P protein has been expressed in the yeast Pichia pastoris. The P703P cDNA encodes a 254-aa polypeptide with an amino-terminal pre-propeptide sequence indicating a potential secretory function. In order to secrete the mature processed P703P protein in the yeast Pichia pastoris, the native P703P secretion signal sequence and the putative pro-peptide coding sequence were replaced by the Saccharomyces cerevisiae alpha pre-pro signal sequence. Mutated P703P (His₄₀→Ala₄₀ at the protease active site) coding sequence was fused to the Saccharomyces cerevisiae alpha prepro signal sequence. The C-terminal part of the recombinant protein was elongated by one glycine and six histidines.

Strain Y1786 was obtained by integrating into the yeast genome 3 to 4 copies of P703P expression cassette plus HIS4 selection gene. On medium containing methanol as carbon source, the P703P protein is efficiently secreted and accumulates into the culture medium with a maximum yield after 96h of induction.

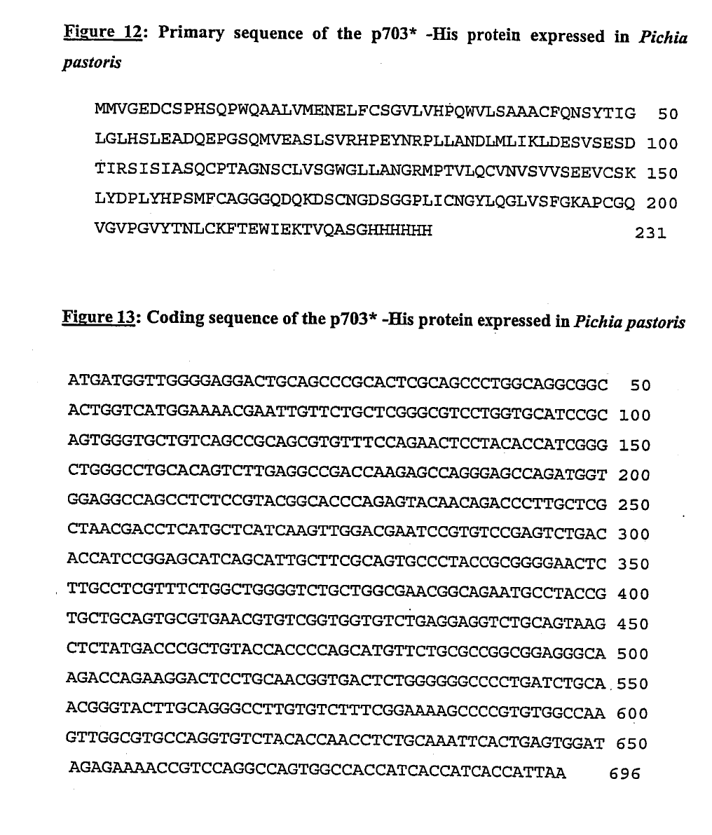

As a result of this cloning strategy, a Pichia recombinant strain, Y1786, has been engineered which carries in his genome expression cassettes of P703P mutated gene. The amino-acid sequence of the secreted recombinant mutated protein and the corresponding nucleotide sequence are described in FIGS. 12 and 13, respectively.

2.—Construction of the Integrative Vector pRIT 15043 and Transformation of the Pichiia pastoris Strain GS115

The starting material was the recombinant plasmid pRIT 14950 (see FIG. 4). This plasmid contains, inserted between the Nco I and Spe I sites in the polylinker of the LITMUS 28 E.coli plasmid, the mutated P703P coding sequence.

In pRIT 14950, the P703P coding sequence is not complete. The N-terminal portion containing the signal peptide and the pro-peptide is not recovered. However, based on prediction of cleavage site for the mature form of the P703P protein, pRIT 14950 contains the entire mature form with start at Valine 5.

The cloning strategy followed to construct the recombinant plasmid pRIT15043 and the integration of expression cassettes into the Pichia genome included the following steps:

a) PCR amplification of the P703 sequence with primers 703P5 and 703P3. Xho I and Not I restriction sites were introduced at respectively the 5′ and 3′ ends of the PCR fragment. The template for the PCR reaction was the pRIT14950 plasmid.

Oligonucleotide sense 703P5:

5′-GAC CGC TCG AGA AAA GAA TGA TGG TTG GGG AGG ACT GC-3′

Oligonucleotide antisense 703P3:

5′-GCG TAC GCG GCC GCT TAA TGG TGA TGG TGA TGG TGG CCA CTG GCC TGG ACG OT-3′

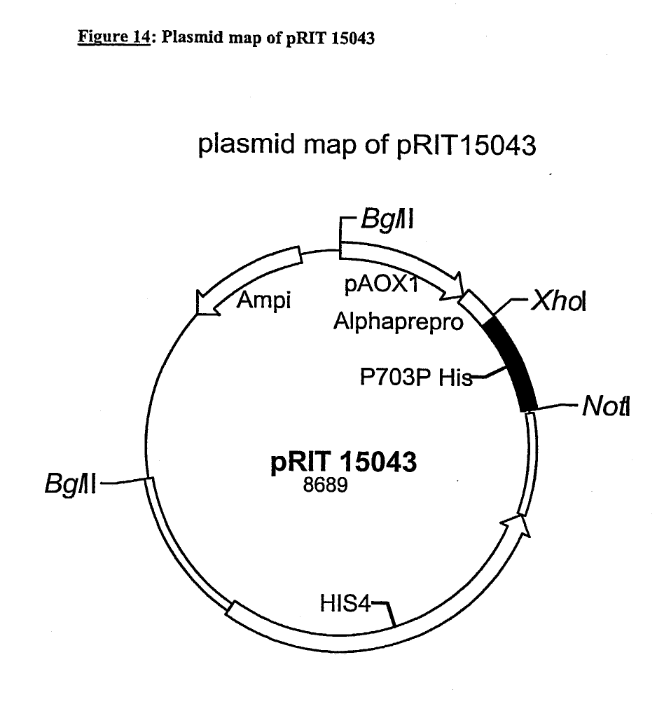

b) The PCR fragment obtained and the commercial plasmid pPIC9 (INVITROGEN) were both digested by Xho I and Not I restriction enzymes, purified on an agarose gel, ligated and transformed in competent XL1-blue cells. The resulting recombinant plasmid received, after verification of the P703P amplified sequence by automatic sequencing, the pRIT15043 denomination (see FIG. 14).

c) Digestion of pRIT 15043 with Bgl II restriction enzyme, purification of the Bgl II fragment carrying the P703P expression cassette plus the HIS 4 gene and transformation of GS115 Pichia recipient strain. This strain is derived from NRRL-Y 11430 (Northern Regional Research Laboratories, Peoria, Ill.) and carries the his4 auxotrophic mutation.

d) To identify multi-copy recombinant strains that exist at a low frequency within HIS4⁺ transformed cell populations, large number of individual transformants (100 colonies minimum) were screened by colony “dot blot” hybridization with DNA probe specific to P703P sequence. Six candidates showing a good signal in dot blot hybridization were selected and screened for product levels by SDS-PAGE and immunoblotting. Using this approach, strain Y1786 has been retained.

The primary structure of the resulting protein has the sequence described in FIG. 12. The coding sequence corresponding to the above protein is illustrated in FIG. 13.

### EXAMPLE VIII

Expression and Characterization of the Recombinant Mutated Protein Produced by Pichia Strain Y1786

1.—Induction Conditions

Y1786 was grown, at 30° C., in Buffered Glycerol-complex Medium (BMGY) to an O.D._{260 nm} of 1-2. Then cells were harvested and resuspended in 1/10 of the original culture volume in Buffered Methanol-complex Medium (BMMY—1% methanol) and incubated (30° C.) for 4 days. An additional 0.5% methanol was added every 24 hours.

2.—Detection of P703P Protein in the Cell-Free Supernatants

The recombinant protein secreted and accumulated in the medium with an optimal secretion after a 96 h induction. On Daiichi stained SDS-PAGE gels, P703P recombinant protein appears as one major band at ±28 kDa (fitting with calculated M.W. of 24.8 kDa and one potential glycosylation site). Two additional bands could correspond to a dimer and a trimer of the protein. Western blot analysis, performed with the monoclonal antibody anti penta-his (QIAGEN), a rabbit polyclonal anti-P703P (1/100) from CORIXA and a rabbit polyclonal anti NS1/P703P (1/1000) from SB, show that all three antibodies recognize a unique band at 28 kDa.

Using standard amounts of purified NS1-P703P-His protein, the yield of secreted native P703P protein was estimated at 10 mg/liter culture supernatant (O.D.30/ml), after 4 days of induction in shake flask condition.

3.—Purification Process

a) Introduction

The purification scheme consists of the following sequence of steps: Culture supernatant→filtration/0.2 μm→IMAC→IEC→UF→sterile filtration. By contrast to the E. coli process, no chaotropic agent or detergent was used along the process. This protocol does not include a reduction/carboxyamidation of the molecule during the purification process but this step has been performed at the end of the purification process. A control run has been made which did not include the reduction/carboxyarnidation. The purified protein, either carboxyamidated or not, was used in the immunological experiment described in Example V, second experiment. Estimated purification yield is around 50 mg purified material/L culture supernatant. Solubility and stability of the purified material were good.

b) Description of the Process

After thawing overnight, the fermentation supernatant [1 liter] was filtered through 0.2 μm filter (Millipak 100, Millipore). The filtrate was perfectly limpid. The filtered supernatant was subjected to IMAC (Ni-Chelating-Sepharose FF, Pharmacia). The column (XK50, Pharmacia; 100 ml) was first equilibrated with PBS buffer pH 7.5. After the sample loading, the column was washed with the same buffer. The protein was then eluted in the previous buffer with a 20 CV linear gradient of increasing imidazole concentration (from 0 to 200 mM). Fractions shown positive for P703P*-His were pooled after SDS-PAGE analysis.

Before continuing the anion exchange chromatography, the conductance of the IMAC-eluate was reduced to around 4 mS/cm with 20 mM phosphate pH 7.5 buffer. The packed bed support (Q-Sepharose FF—XK50 column, 60 ml, Pharmacia) was first equilibrated with 20 mM phosphate pH 7.5 buffer. After the sample loading, a washing step was performed with the same buffer. The protein was then eluted with the same buffer containing a 20 CV linear gradient of increasing NaCl concentration (from 0 to 500 mM). Fractions shown positive for P703P*-His were pooled after SDS-PAGE analysis.

The Q-Sepharose FF-eluate was then concentrated and diafiltered against the 20 mM phosphate pH 7.5 buffer in an tangential flow filtration unit (Labscale, Millipore) equipped with a PLCGC 10 Kd cut-off membrane of 50 cm² (Pellicon XL, Millipore). Ultrafiltration retentate containing P703P*-His was sterile filtered through 0.22 μm membrane (Millex GV, Millipore).

As a loss of product was observed in the permeate of this first ultrafiltration, a concentration of the permeate was performed in a stirred cell device (Amicon) equipped with a Omega 5 Kd cut-off membrane (Filtron) to recover the material. The concentrated permeate was dialysed (in visking tubing) against the 20 mM phosphate pH 7.5 buffer and was also sterilized by filtration through 0.22 μm membrane (Millex GV, Millipore).

b) Reduction/Carboxyamidation Step

The purified antigen was diluted with an equivalent volume of 20 mM P0₄ buffer-8 M GuHCl—1% Empigen BB-40 mM Glutathion pH 7.5 and left at room temperature in the dark under gentle agitation for 1 hour. The carboxyamidation of the protein was then performed by addition of iodoacetamide up to a final concentration of 100 mM and adjustment of the pH to 7.5 with concentrated NaOH solution. The mixture was left at room temperature in the dark under gentle agitation for 30 minutes. After addition of 0.2% Tween 80 (final concentration), the sample was dialysed 18 hours at +4° C. against 20 mM Tris buffer −0.2% Tween 80 pH 8.0 and sterilised by filtration through 0.22 μm membrane.

### CONCLUSION

We have demonstrated that the chemically modified prostase is immunogenic in mice, and that this immunogenicity (antibody response) can be further increased by the use of the adjuvant described above.

We have demonstrated that the fusion protein NS1-P703P*-His, when purified using the optimised process described in the invention, can be produced at a high yield and with consistency, both compatible with an up-scaleable industrial process. We have further demonstrated that the process of the invention leads to a less aggregated, less oxidised, more soluble and more stable recombinant protein as compared to a process not involving the reduction/carboxyamidation step. Purification can be enhanced by derivatising the thiols that form disulphide bonds.

We have also demononstrated that the reduction/carboxyamidation step could alternatively be performed at the end of the purification process.

### REFERENCES

Abbas F., Scardino P. “The Natural History of Clinical Prostate Carcinoma.” Cancer 80:827-833 (1997)

Bostwick D., Pacelli A., Blute M. et al. “Prostate Specific Membrane Antigen “Expression in Prostatic Intraepithelial Neoplasia and Adenocarcinoma” Cancer 82:2256-2261 (1998)

Frydenberg M., Stricker P., Kaye K. “Prostate Cancer Diagnosis and Management.” The Lancet 349:1681-1687 (1997)

C. Hackett, D. Horowitz, M. Wysocka & S. Dillon, J. Gen. Virology, 73, 1339-1343 (1992)

Kensil C. R., Soltysik S., Patel U., et al. in: Channock R. M., Ginsburg H. S., Brown F., et al., (eds.), Vaccines 92, (Cold Spring Harbor Laboratory Press, Cold Spring Harbor, N.Y.), 36-40: (1992)

Nelson P., Lu Gan, Ferguson C., Moss P., Gelinas R., Hood L. & Wand K., “Molecular cloning and characterisation of prostase, an androgen-regulated serin protease with prostate restricted expression”, Proc. Natl. Acad Sci. USA 96, 3114-3119 (1999)

Pound C., Partin A., Eisenberg M. et al. “Natural History of Progression after PSA Elevation following Radical Prostatectomy.” Jama 281:1591-1597 (1999)

Ribi E., et al. in: Levine L., Bonventre P. F., Morello J., et al. (eds)., American Society for Microbiology, Washington D.C., Microbiology 1986, 9-13; (1986)

Xue B H., Zhang Y., Sosman J. et al. “Induction of Human Cytotoxic T-Lymphocytes Specific for Prostate-Specific Antigen.” Prostate 30(2):73-78 (1997)

1. A prostase protein antigen, fragment or homologue thereof comprising derivatised thiol residues.

2. An antigen as claimed in claim 1 wherein the prostase derivatised thiol residues are alkylated.

3. An antigen as claimed in claim 2 wherein the prostase derivatised thiol residues are carboxyalkylated.

4. An antigen as claimed in claim 3 wherein the prostase derivatised thiol residues are carboxyamidated or carboxymethylated.

5. An antigen as claimed in any of claims 1 to 4 wherein the prostase is selected from the group consisting of SEQ ID NO:5, SEQ ID NO:6, SEQ ID NO:7, and SEQ ID NO:8.

6. An antigen as claimed in any of claims 1 to 5 wherein a mutation is introduced into the active site.

7. A fusion protein comprising an antigen as claimed in any of claims 1 to 6 wherein the antigen is linked to a fusion partner.

8. A fusion protein as claimed in claim 7 wherein the fusion partner is an immunological fusion partner or an expression enhancer fusion partner.

9. A fusion protein as claimed in claim 8 wherein the fusion partner is NS1 protein from influenza or a fragment thereof.

10. An antigen as claimed in any of claims 1 to 9 wherein the antigen further comprises an affinity tag.

11. A vaccine containing a protein as claimed in any of claims 1 to 10.

12. A vaccine as claimed in claim 11 additionally comprising an adjuvant, and/or immunostimulatory cytokine or chemokine.

13. A vaccine as claimed in claim 11 or 12 wherein the adjuvant comprises one or more of 3D-MPL, QS21, a CpG oligonucleotide or a polyethylene ether or ester.

14. A vaccine as claimed in claim 11 or 13 wherein the protein is presented in an oil in water or a water in oil emulsion vehicle.

15. A vaccine as claimed in any of claims 10 to 14 additionally comprising one or more other antigens.

16. A vaccine as claimed herein for use in medicine.

17. Use of a protein as claimed herein for the manufacture of a vaccine for immunotherapeutically treating a patient suffering from prostate cancer or other prostase-associated tumours.

18. A process for the purification of a prostase antigen, fragment, fusion or homologue thereof, comprising solubilising the protein utilising a combination of both a strong chaotropic agent and a detergent.

19. A process for the production of a a prostase antigen, fragment, fusion or homologue thereof, comprising the steps of treating the protein to reduce the protein's intra- and inter-molecular disulphide bonds, and blocking the thiol to prevent oxidative recoupling.

20. A process for the purification of a prostase antigen, fragment, fusion or homologue thereof comprising solubilising said antigen, fragment, fusion or homologue, reducing at least one of the protein's intra- and inter-molecular disulfide bond utilising a reducing agent, filtering the product, and blocking the resulting free thiol group.

21. A process according to claims 18 to 20, further comprising the step of subjecting the protein to one or more chromatographic steps.

22. A process as claimed in claim 21 wherein the chromatographic step involves subjecting the protein to IMAC chromatography at a pH between 7.5 and 10.

23. A process as claimed in claims 19 to 22 wherein the blocking agent is selected to induce a stable covalent derivative.

24. A process as claimed in claim 23 wherein the blocking agent is an alpha haloacid or alpha haloamide.

25. A process as claimed in claim 24 wherein the blocking agent is iodoacetic acid or iodoacetamide.

26. A process for the production of a vaccine, comprising the steps of purifying a prostase protein or a derivative thereof, by the process of any of claims 18 to 25 and admixing the resulting protein as claimed herein with a suitable adjuvant, diluent or other pharmaceutically acceptable excipient.

27. A method of treating patients susceptible to or suffering from prostate-cancer comprising administering to said patients a pharmaceutically active amount of the vaccine according to claims 11 to 16.
